(19) United States
(12) Patent Application Publication
(10) Pub. No.: US 2025/0297333 A1
(43) Pub. Date: Sep. 25, 2025

(54) SYSTEMS AND METHODS FOR DETECTION OF MICROBIAL NUCLEIC ACIDS

(71) Applicant: University of Connecticut, Farmington, CT (US)

(72) Inventors: Changchun Liu, Farmington, CT (US); Jiongyu Zhang, New Britain, CT (US)

(21) Appl. No.: 19/083,639

(22) Filed: Mar. 19, 2025

Related U.S. Application Data
(60) Provisional application No. 63/567,255, filed on Mar. 19, 2024.

(51) Int. Cl. C12Q 1/70; C12Q 1/44; C12Q 1/689; C12Q 1/6895

(52) U.S. Cl. CPC C12Q 1/703; C12Q 1/44; C12Q 1/689; C12Q 1/6895; C12Q 1/708

(57) ABSTRACT

This disclosure provides compositions, methods, and systems comprising a “Universal Nuclease for Identification of Virus Empowered by RNA-sensing” (UNIVERSE) assay. The compositions, methods, and systems find use in various settings including the clinical detection of pathogen nucleic acids.

### CROSS-REFERENCE TO RELATED APPLICATIONS

This application claims the benefit of U.S. Provisional Application No. 63/567,255 filed Mar. 19, 2024, the contents of which are incorporated by reference herein.

### STATEMENT REGARDING FEDERALLY SPONSORED RESEARCH OR DEVELOPMENT

This invention was made with government support under CA269147 and AI154642 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 38,709 Byte XML file named “UCONN_44804_202_SequenceListing.xml,” created on Mar. 19, 2025.

### FIELD

This disclosure provides compositions, methods, and systems comprising a “Universal Nuclease for Identification of Virus Empowered by RNA-sensing” (UNIVERSE) assay. The compositions, methods, and systems find use in various settings including the clinical detection of pathogen nucleic acids.

### BACKGROUND

CRISPR (Clustered Regularly Interspaced Short Palindromic Repeats) technology, widely recognized as a groundbreaking genome editing tool, has shown potential in nucleic acid-based molecular diagnostics. Among the CRISPR-Cas families, Cas12a displays a distinctive trans-cleavage activity documented to result in massive indiscriminate degradation of single-stranded DNA substrate targets. For example, Cas12a displays trans-cleavage of DNA substrates labeled with a fluorophore and quencher pair (ssDNA-FQ), when a DNA sequence complementary to the spacer of CRISPR (crRNA) is recognized.

However, Cas12a-based detection of double-stranded DNA (dsDNA) targets is inherently limited by a protospacer adjacent motif (PAM) sequence (e.g., TTTV) responsible for facilitating the separation of dsDNA and subsequent crRNA invasion. In contrast, the PAM sequence is not essential for ssDNA detection. Recently, a series of suboptimal PAMs (e.g., VTTV, TCTV, and TTVV) were discovered to generate an equivalent or even greater trans-cleavage fluorescence response compared to the canonical PAM under conventional CRISPR-Cas12a assay protocols using LbCas12a. However, commonly used Cas12a orthologs including LbCas12a, AsCas12a and FnCas12a all lack the ability to detect a random sequence within a dsDNA amplicon unless additional steps are taken such as artificially introducing a PAM sequence through amplification, carefully designing a strand displacement reaction pathway, utilizing nucleases that degrade dsDNA to ssDNA, or implementing target-dependent synthesis of a crRNA matching an effective activator introduced to the reaction. These required additional steps significantly limit the selection of target sequences and reduce the detection efficiency in clinical diagnostic applications of Cas12a.

### SUMMARY

The disclosure provides, in some embodiments, a nucleic acid detection assay, and systems comprising same, comprising CRISPR-Cas12a and a T7 transcription step that converts nucleic acid targets lacking a protospacer adjacent motif (PAM) into single stranded RNA (ssRNA) targets (e.g., thereby circumventing the requirement of conventional CRISPR assays and systems that require a target substrate contain a PAM sequence).

For example, in some embodiments, the disclosure provides a nucleic acid detection assay comprising a Cas12a protein exhibiting trans-cleavage activity activated by RNA target polynucleotide. In some embodiments, the Cas12a protein exhibiting RNA-activating activity possesses DNA-activating activity. In further embodiments, the Cas12a protein displays negligible degradation of RNA targets. In some embodiments, the Cas12a protein is AsCas12a or AsCas12a variant (e.g., AsCas12a V3 or AsCas12a Ultra). In other embodiments, the Cas12a protein is LbCas12a. In some embodiments, the Cas12a protein (e.g., activated by RNA target) exhibits greater specificity for targets comprising one or more mutations compared to DNA activation (e.g., a Cas12a protein of the assay discriminates more effectively between perfectly matched and mutated targets compared to conventional CRISPR assays based on DNA activation (e.g., thereby reducing off-target effects)). For example, in some embodiments, the assay differentiates targets from non-targets based on single base pair differences. In some embodiments, the assay further comprises recombinase polymerase amplification (RPA) and T7 transcription. In some embodiments, the assay reliably detects PAM-less sequences that are undetectable by conventional RPA/CRISPR-Cas12a assays (e.g., the assay detects any known target nucleic acid sequence (DNA or RNA) thereby overcoming one of the major limitations of conventional Cas12a assays that requires a PAM near the target site to enable strand separation and crRNA binding). In some embodiments, the assay is used for human diagnostic testing (e.g., for cancer, disease, or other health status) and/or microbial pathogen (e.g., pathogenic virus, bacteria, fungi, or protozoa) detection. The assay is not limited to these particular uses. Indeed, the assay finds use in numerous settings including those described herein. In some embodiments, the assay displays greater specificity and/or sensitivity than conventional RPA/CRISPR-Cas12a assays. In other embodiments, the assay displays enhanced detection efficiency compared to conventional RPA/CRISPR-Cas12a assays (e.g., the assay provides a robust diagnostic with sensitivity in the attomolar range). For example, the UNIVERSE assay provides a robust diagnostic with sensitivity with about 1-5 attomolar sensitivity, about 1-4 attomolar sensitivity, about 2-4 attomolar sensitivity, or about 3-4 attomolar sensitivity. In some embodiments, the UNIVERSE assay provides a robust CRISPR-Cas12a-based diagnostic with about 3-3.5 attomolar sensitivity. The assay, in some embodiments and for ease of reference, is referred to as “Universal Nuclease for Identification of Virus Empowered by RNA-Sensing” (“UNIVERSE” or “UNIVERSE ASSAY” or the like).

Accordingly, the disclosure provides, in some embodiments, a method of detecting target nucleic acids (e.g., a microbial nucleic acid, a cancer gene nucleic acid, or other target described herein) in a sample using UNIVERSE. In some embodiments, UNIVERSE comprises the steps of: amplification of target nucleic acids, if present, via recombinase polymerase amplification (RPA); transcription of the RPA amplicons into ssRNA targets; and activation of CRISPR-Cas12a via the ssRNA targets. In some embodiments, the method comprises contacting a sample suspected of containing target nucleic acids with recombinase polymerase and reagents for amplification of the target nucleic acids; amplifying the target nucleic acids in the sample via recombinase polymerase amplification (RPA) to generate amplicons of the target nucleic acids (e.g., amplicons of the microbial nucleic acid, amplicons of the cancer gene nucleic acid, or amplicons of the other targets described herein); transcription of the amplified target nucleic acids (e.g., transcription of the amplicons of the microbial nucleic acid, transcription of the amplicons of the cancer gene nucleic acid, or transcription of the amplicons of the other targets described herein) into ssRNA targets using T7 RNA polymerase; activation of CRISPR-Cas12a by the ssRNA targets; and generating a detectable signal via CRISPR-Cas12a trans-cleavage of reporter molecules (e.g., a ssDNA reporter tagged with a fluorophore and quencher (e.g., SEQ ID NO. 5) and/or a ssRNA reporter tagged with a fluorophore and quencher (e.g., SEQ ID NO. 6)); wherein the presence of signal indicates the presence of the target nucleic acids in the sample, and the absence of signals indicates the absence of the target nucleic acids in the sample. In some embodiments, amplification of target nucleic acids comprises use of a primer (e.g., forward primer) comprising a T7 promoter sequence (e.g., GAAATTAATACGACTCACTATAGGG (SEQ ID NO: 42)). In some embodiments, the amplified target nucleic acids and/or ssRNA targets produced from same (e.g., via T7 RNA polymerase transcription) lack a PAM sequence. The disclosure is not limited by the type of sample. Indeed, a variety of samples may be used including but not limited to blood, serum, plasma, saliva, urine, vaginal fluid, semen or other sample described herein. In some embodiments, the method comprises extracting and/or purifying nucleic acids from the sample. In some embodiments, the method comprises the step of reverse transcription to generate cDNA prior to the RPA amplification step (e.g., if starting with an RNA sample). In other embodiments, the steps of reverse transcription and RPA amplification are carried out at the same time (e.g., in the same vessel or tube). In some embodiments, the method uses one or more control target sequences (e.g., synthetic and/or spiked RNA or DNA control target sequence (e.g., to monitor and/or verify reaction efficiency and/or to act as positive and/or negative controls)). In some embodiments, the method is used to amplify, transcribe, and detect a plurality of different target nucleic acids (e.g., amplicons of a plurality of different microbial nucleic acids, amplicons of a plurality of different cancer gene nucleic acids, or amplicons of a plurality different other targets described herein) simultaneously (e.g., multiplexing and/or parallel detection of different pathogens and/or genetic markers). In some embodiments, the method is automated or semi-automated via incorporation of s microfluidic device. In other embodiments, the method is utilized in a point of care platform known in the art. In some embodiments, lateral flow detection and/or a secondary fluorescence readout is used to validate or confirm the robustness of the method (e.g., to validate and/or confirm the integrity of the amplification, transcription, and/or detection steps). The disclosure is not limited by the way in which a method of detecting target nucleic acids in a sample is used. For example, the method of detecting target nucleic acids in a sample may be used in a nucleic acid detection system and/or as a diagnostic or in a diagnostic device, or in another way described herein or known in the art. In some embodiments, the method provides higher signal intensities and lower detection limits in clinical samples compared to existing, conventional assays. In some embodiments, the method provides enhanced detection of target nucleic acids compared to conventional RPA/CRISPR-Cas12a assays (e.g., provides about a ten to one-hundred (10-100)-fold improvement over detection of target nucleic acids compared to conventional RPA/CRISPR-Cas12a assays). For example, in some embodiments, when using the CRISPR-Cas12a nuclease LbCas12a Ultra, the method displays a detection limit of about 3 copies/μL (e.g., compared to a detection limit of about 300 copies/μL using LbCas12a Ultra in a conventional RPA/CRISPR-Cas12a assay). In other embodiments, when using the CRISPR-Cas12a nuclease AsCas12a, the method displays a detection limit of about 30 copies/μL (e.g., compared to a detection limit of about 300 copies/μL using AsCas12a in a conventional RPA/CRISPR-Cas12a assay). In some embodiments, RNA activated Cas12a of the method reduces false-positive signals and/or improves mismatch discrimination compared to conventional RPA/CRISPR-Cas12a assay (e.g., reduces background signal/noise observed with DNA targets in conventional RPA/CRISPR-Cas12a assays).

In some embodiments, the disclosure provides a method for detecting a microbial nucleic acid in a sample, which method comprises: obtaining a sample suspected of containing the microbial nucleic acid from a subject (e.g., a human subject); generating amplicons of the microbial nucleic acid via contacting the sample suspected of containing the microbial nucleic acid with recombinase polymerase and reagents for amplification of the microbial nucleic acid and amplifying the microbial nucleic acid via recombinase polymerase amplification (RPA), wherein the amplicons of the microbial nucleic acid lack protospacer adjacent motif (PAM) sequences; transcribing the amplicons of the microbial nucleic acid into single-stranded RNA (ssRNA) targets using T7 RNA polymerase; incubating the ssRNA targets with a CRISPR-Cas12a nuclease, one or more guide RNA sequences specific for one or more regions within the ssRNA targets, and reporter molecules (e.g., under conditions sufficient to allow binding of the one or more guide RNA sequences to one or more regions within the ssRNA targets); activating the CRISPR-Cas12a nuclease via binding of the one or more guide RNA sequences to the one or more regions within the ssRNA targets; and generating a detectable signal via CRISPR-Cas12a trans-cleavage of the reporter molecules. In some embodiments, each of the above steps occur in the same tube and/or vessel. In some embodiments, nucleic acid is extracted and/or purified from the sample (e.g., prior to generating amplicons of the microbial nucleic acid). In some embodiments, the presence of a detectable signal indicates the presence of the microbial nucleic acid in the sample. In some embodiments, the absence of detectable signal indicates the absence of the microbial nucleic acid in the sample. In some embodiments, the subject is a mammal such as human, cattle, cow, dog, cat, or pig. In some embodiments, the subject is human. In some embodiments, generating amplicons of the microbial nucleic acid comprises use of a primer comprising a T7 promoter sequence. In some embodiments, the microbial nucleic acid is DNA. In other embodiments, the microbial nucleic acid is RNA. In some embodiments, microbial nucleic acid comprises DNA and RNA. In some embodiments, reverse transcription is utilized to generate cDNA. In some embodiments, reverse transcription and generating amplicons of the microbial nucleic acid occur at the same time (e.g., in the same vessel or tube). In some embodiments, the method detects attomolar amounts of microbial nucleic acid. For example, in some embodiments, the method detects with about 1-5 attomolar sensitivity, about 1-4 attomolar sensitivity, about 2-4 attomolar sensitivity, or about 3-4 attomolar sensitivity. In some embodiments, the method detects about 3-3.5 attomolar sensitivity. In some embodiments, activating the CRISPR-Cas12a nuclease does not occur when there is a mismatch between the ssRNA targets and the guide RNA sequences. The method is not limited by the type or amount of mismatch. In some embodiments, the mismatch is a single base-pair difference. In other embodiments, the mismatch is a two, three, four, five, six, seven, eight, nine, ten, or more base-pair mismatch. In some embodiments, activating the CRISPR-Cas12a nuclease occurs via binding of the one or more guide RNA sequences to the one or more regions within the ssRNA targets. The method is not limited by the type of sample. Indeed, a variety of samples may be used including but not limited to blood, mucous, serum, plasma, saliva, urine, stool, vaginal fluid, synovial fluid, spinal fluid, and/or semen. In some embodiments, the sample is an upper respiratory sample (e.g., obtained from a nasopharyngeal swab, oropharyngeal (throat) swab, mid-turbinate nasal swab, anterior nasal swab, nasopharyngeal wash/aspirate and/or nasal aspirate). The method is not limited by the reporter molecule used. A variety of reporter molecules are known in the art and can be used in methods disclosed herein including but not limited to fluorophore-quencher reporters (e.g., single-stranded DNA (ssDNA) probe tagged with a fluorophore and quencher). The method is not limited by the type of microbial nucleic acid detected. For example, microbial nucleic acid may be from any one or more pathogenic viruses, bacteria, protozoa, and/or fungi described herein. The method is not limited by the CRISPR-Cas12a nuclease used. Exemplary CRISPR-Cas12a nucleases include AsCas12a, AsCas12a V3, AsCas12a Ultra, LbCas12a, and/or combinations thereof. In some embodiments, the method detects as little as about 3 copies/μL. In some embodiments, one or more control sequences are utilized to monitor and/or verify reaction efficiency and/or to act as positive and/or negative controls. In some embodiments, the control sequence is a synthetic and/or spiked RNA or DNA control target sequence. The method may be used for the detection of nucleic acid from a single microbe, or it may be used for the parallel, simultaneous detection of nucleic acid (e.g., ssRNA targets generated according to the disclosed method) from a plurality of different pathogenic microbes. Indeed, the method may be used to detect nucleic acid (e.g., ssRNA targets generated according to the method) of a plurality of different viruses, bacteria, protozoa, and/or fungi. In some embodiments, the activated CRISPR-Cas12a nuclease does not cleave the ssRNA targets. The method may be utilized in a variety of ways and settings. For example, in some embodiments, the method is used in a lateral flow immunochromatographic assay. In some embodiments, the method is performed more than one time from different samples obtained from the same subject over a period of time.

The disclosure also provides kits for detecting a microbial nucleic acid in a sample comprising recombinase polymerase; a primer comprising a T7 promoter sequence and other reagents for amplification of the microbial nucleic acid sufficient to generate amplicons of the microbial nucleic acid lacking protospacer adjacent motif (PAM) sequences; T7 RNA polymerase for transcribing amplicons of the microbial nucleic acid into single-stranded RNA (ssRNA) targets; CRISPR-Cas12a nuclease; one or more guide RNA sequences specific for one or more regions within the ssRNA targets; and reporter molecules. In some embodiments, all or some of the kit components are lyophilized. In some embodiments, the reporter molecules comprise SEQ ID NO. 5 and/or SEQ ID NO. 6. In some embodiments, the primer comprising a T7 promoter sequence comprises SEQ ID NO. 11, SEQ ID NO. 16, SEQ ID NO. 24, and/or SEQ ID NO. 33. In some embodiments, other reagents for amplification of the microbial nucleic acid sufficient to generate amplicons of the microbial nucleic acid lacking protospacer adjacent motif (PAM) sequences comprise SEQ ID NO. 12, SEQ ID NO. 17, SEQ ID NO. 25, and/or SEQ ID NO. 34. In some embodiments, the one or more guide RNA sequences comprise SEQ ID NO. 13, SEQ ID NO. 18, SEQ ID NO. 26, SEQ ID NO. 35, SEQ ID NO. 36, SEQ ID NO. 37, SEQ ID NO. 38, SEQ ID NO. 39, and/or SEQ ID NO. 40. In some embodiments, the control target sequence comprises SEQ ID NO. 14, SEQ ID NO. 19, SEQ ID NO. 27, SEQ ID NO. 28, or SEQ ID NO. 41.

In one embodiment, the disclosure provides a method of detecting, diagnosing, and/or identifying a disease or disease state in a subject comprising detecting target nucleic acids in a sample from the subject using the UNIVERSE assay. The disclosure is not limited by the disease or disease state detected and/or diagnosed in a subject. Indeed, the UNIVERSE assay can be used to detect and/or diagnose a variety of diseases, disorders, and/or disease states. In some embodiments, the disease or disorder is a pulmonary disease or disorder, kidney disease or disorder, liver disease or disorder, heart disease or disorder, gastrointestinal disease or disorder, heart disease or disorder, blood disease or disorder, lymphatic disease or disorder, brain or neurological disease or disorder, respiratory disease or disorder, cancer, blood disease or disorder, immune system disease or disorder, pregnancy disease or disorder, endocrine disease or disorder, nervous system disease or disorder, organ disease or disorder, an autoimmune disease or disorder, infection, or other disease or disorder described herein. Indeed, any disease or disorder for which a known nucleic acid sequence can be used to detect the disease, disorder, and/or disease state can be detected using the UNIVERSE assay (e.g., to detect a nucleic acid sequence (e.g., a single nucleotide polymorphism (SNP), a splice variant, a deletion, a frameshift mutation, or other nucleic acid sequence) that is diagnostic for the disease, disorder, or disease state). In some embodiments, the UNIVERSE assay is used to detect the presence of an infectious agent (e.g., detect microbial nucleic acid of a pathogen).

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

FIGS. 1A-H show RNA-initiated nuclease activity with Cas12a. FIGS. 1A-D) Real-time fluorescence of ssRNA-activated trans-cleavage with FIG. 1A) AsCas12a V3 (IDT), FIG. 1B) AsCas12a Ultra (IDT), FIG. 1C) LbCas12a Ultra (IDT), and D) EnGen Lba Cas12a (NEB). For FIGS. 1A-D), experiments at each concentration were run in triplicate and graphs represent means (bold line)+standard deviation (s.d.). FIG. 1E) Comparison of trans-cleavage activity among the enzymes tested for different types of targets and reporters. FIG. 1F) Denaturing PAGE gel electrophoresis for identification of cis-cleavage and trans-cleavage products using 5′ FAM-labeled ssRNA target with a 5′ extension. FIG. 1G) Denaturing PAGE gel electrophoresis for identification of cis-cleavage and trans-cleavage products using 5′ FAM-labeled ssRNA target with both 5′ and 3′ extensions. FIG. 1H) A summarization of nuclease activity of LbCas12a, AsCas12a, and SuCas12a2 in terms of the types of targets, cis-cleavage substrates, and trans-cleavage substrates.

FIGS. 2A-E show a specificity comparison of trans-cleavage activity with Cas12a by ssDNA and ssRNA targets. FIG. 2A) Schematic illustration of ssDNA- and ssRNA-activated trans-cleavage activity of Cas12a and the mutated target sequences used to investigate the specificity of trans-cleavage activity with ssDNA and ssRNA targets. Wild-type (WT) sequences refer to target sequence fully complementary to the spacer of crRNA. Mutation position is indicated by the notation for every mutant and highlighted in pale grey. FIGS. 2B-E, Measurements were carried out with FIG. 2B) AsCas12a V3 (IDT), FIG. 2C) AsCas12a Ultra (IDT), FIG. 2D) LbCas12a Ultra (IDT), and FIG. 2E) EnGen Lba Cas12a (NEB) by calculating the relative fluorescence enhancement rate to that for the fully matched target. The error bars represent the means±standard deviation (s.d.) from three replicates. The experimental groups with negative fluorescence enhancement rates, indicating no reactivity to a mismatched target, were left blank in the figure.

FIGS. 3A-H show the UNIVERSE assay for highly sensitive PAM-free nucleic acid detection. FIG. 3A, Schematic illustration of the UNIVERSE assay for detection of a sequence lacking PAM. In the RPA step, T7 promoter was brought into the amplicon by prefixing it to the forward primer. T7 RNAP recognizes the T7 promoter in RPA amplicons and starts synthesizing RNA strands, which include a 20-nt target sequence complementary to the spacer of crRNA and can be released in single-stranded form for the downstream CRISPR reaction. If directly using the RPA amplicon (dsDNA target) in the conventional CRISPR reaction, the fluorescence signal is much weaker than that of T7-transcribed amplicons (RNA target) since the target lacks PAM. FIG. 3B, Denaturing PAGE gel electrophoresis for identification of the RPA amplicons and T7-transcribed amplicons. The template is HIV p24 plasmid at two different concentrations. The position of the full-length RPA amplicon and transcription product, which are expected to be 161 nt, are determined by the position of the ssDNA ladder and marked in colored boxes. C-D, Real-time fluorescence kinetics of detection with FIG. 3C) the UNIVERSE assay and FIG. 3D) the conventional RPA/CRISPR-Cas12a assay. FIGS. 3E-F, Endpoint fluorescence enhancement at 120 min for detection with FIG. 3E) the UNIVERSE assay and FIG. 3F) the conventional RPA/CRISPR-Cas12a assay. The detection was performed on 10-fold serially diluted HIV p24 plasmid starting at 3×10⁶ copies/μL. The inset figures are zoomed-in views when template concentrations were low. The error bars represent the means±standard deviation (s.d.) from four replicates. The unpaired two-sample t-test was used to analyze the statistical significance. ****: p<0.0001. ns: not significant. FIG. 3G, Comparison of fluorescence enhancement rates in detection of HIV p24 plasmid using the UNIVERSE assay with T7 RNAP and the conventional RPA/CRISPR-Cas12a assay without T7 RNAP. The logarithm of fluorescence enhancement rates to base 10 was used for a clearer comparison when the template concentration was low. FIG. 3H, Fluorescence image of reactions of the UNIVERSE assay and conventional RPA/CRISPR-Cas12a assay using LbCas12a Ultra (IDT). The image was captured after a 120-min reaction. The concentrations of 10-fold serially diluted HIV p24 plasmid template are marked in red with a unit of “copies/μL.” The experiment was run in quadruplicate for each concentration of the assay.

FIGS. 4A-I show that UNIVERSE permits detection of sequences that are undetectable with conventional RPA/CRISPR-Cas12a assays. FIG. 4A, Locations of the crRNA targeting sites in the amplicon of the HIV RNA used for the detection. The pale grey bases refer to a segment of targeting sites for RPA primers. Six crRNA targeting sites were selected in the black amplicon sequence and marked with their indexes. FIGS. 4B-G, Real-time fluorescence detection with UNIVERSE and conventional RT-RPA/CRISPR-Cas12a using the six selected crRNAs and the fold change of fluorescence enhancement with 3 h of reaction. The figures indicate the results for B, crRNA-1; C, crRNA-2; D, crRNA-3; E, crRNA-4; F, crRNA-5; and G, crRNA-6. The error bars represent the means±standard deviation (s.d.) from four replicates. FIG. 4H, Degree of consensus for every CRISPR target in the amplicon with the location of selected targets marked in the figure. The degree of consensus was measured by calculating the proportion of 20-nt CRISPR targets completely matching the reference (NC_001802.1), observed in valid RPA amplicons from sequencing records of HIV from the NCBI Virus database. FIG. 4I, Detection comparison of clinical HIV-positive samples using the UNIVERSE versus conventional RT-RPA/CRISPR-Cas12a assays.

FIGS. 5A-I shows clinical validation of the UNIVERSE assay for HIV and HPV 16 detection. FIG. 5A, Cq values for detection of HIV RNA-positive control with the RT-qPCR assay. FIG. 5B, Real-time fluorescence kinetics for detection of HIV RNA-positive control with the UNIVERSE assay. FIG. 5C, Fluorescence enhancement at the endpoint of detection of HIV RNA-positive control with the UNIVERSE assay. ***: p<0.001. ****: p<0.0001. ns: not significant. Copy numbers in A-C were determined by digital RT-PCR. FIG. 5D, Cq values for detection of clinical HIV-positive and HIV-negative samples with the RT-qPCR assay. FIG. 5E, Fluorescence enhancement for HIV clinical sample detection with the UNIVERSE assay after 180 min of incubation. The inset compares fluorescence enhancement of all positive samples to all negative samples. FIG. 5F, Fluorescence image of UNIVERSE reactions for clinical HIV sample detection. The image was captured after 180 min of reaction. The sample names are marked above each set of tubes. For D-F, the positive control (PC) was undiluted HIV RNA-positive control, and the negative control (NC) was extractions from healthy human plasma. G, Cq values for detection of clinical HPV 16 positive and negative samples with the qPCR assay. FIG. 5H, Fluorescence enhancement for HPV 16 clinical sample detection with the UNIVERSE assay after 120 min of incubation. The inset compares fluorescence enhancement of all positive samples to all negative samples. FIG. 5I, Fluorescence image of UNIVERSE reactions for clinical HPV 16 sample detection. The image was captured after 120 min of reaction. The sample names are marked above each set of tubes. For G-I, the positive control (PC) was 1 fM of plasmid containing an HPV 16 L1 genome fragment, and the negative control (NC) was AE buffer from the DNeasy Blood and Tissue Kit used for HPV 16 DNA extraction. The qPCR/RT-qPCR assay was run in triplicate and the UNIVERSE assay was run in quadruplicate for each sample. The error bars represent the means±standard deviation (s.d.) from four replicates for UNIVERSE or three replicates for RT-qPCR. The unpaired two-sample t-test was used to analyze the statistical significance.

FIGS. 6A-E show Michaelis-Menten kinetics of RNA-activating Cas12a. FIG. 6A) Background-subtracted fluorescence F_{cl} versus concentration of cleaved reporters c_{cl}. FIG. 6B) Background-subtracted fluorescence F_{ucl} versus concentration of uncleaved reporters c_{ucl}. FIG. 6C) Michaelis-Menten kinetics of RNA-activated trans-cleavage with AsCas12a V3 (IDT). FIG. 6D) Michaelis-Menten kinetics of RNA-activated trans-cleavage with AsCas12a Ultra (IDT). Three technical replicates were measured for each substrate concentration and the error bars represent means±standard deviation (s.d.). The fitted curves are represented by red solid lines and the fitted parameters are provided in each figure. FIG. 6E) Comparison of parameters in Michaelis-Menten kinetics for different Cas12a variants with varying activator types.

FIGS. 7A-D show real-time fluorescence kinetics of ssDNA-activated trans-cleavage on ssDNA reporters with the following Cas12a enzymes: FIG. 7A) AsCas12a V3 from IDT; FIG. 7B) AsCas12a Ultra from IDT; FIG. 7C) LbCas12a Ultra from IDT; FIG. 7D) EnGen Lba Cas12a from NEB. Experiments at each concentration were performed in triplicate and graphs represent means (bold line)+standard deviation (s.d.).

FIGS. 8A-D shows real-time fluorescence kinetics of ssDNA-activated trans-cleavage on ssRNA reporters with the following Cas12a enzymes: FIG. 8A) AsCas12a V3 from IDT; FIG. 8B) AsCas12a Ultra from IDT; FIG. 8C) LbCas12a Ultra from IDT; FIG. 8D) EnGen Lba Cas12a from NEB. Experiments at each concentration were performed in triplicate and graphs represent means (bold line)+standard deviation (s.d.).

FIGS. 9A-D shows real-time fluorescence kinetics of ssRNA-activated trans-cleavage on ssRNA reporters with the following Cas12a enzymes: FIG. 9A) AsCas12a V3 from IDT; FIG. 9B) AsCas12a Ultra from IDT; FIG. 9C) LbCas12a Ultra from IDT; FIG. 9D) EnGen Lba Cas12a from NEB. Experiments at each concentration were performed in triplicate and graphs represent means (bold line)+standard deviation (s.d.).

FIGS. 10A-D shows fluorescence enhancement rates of ssRNA-activated trans-cleavage on ssDNA reporters with Cas12a enzymes: FIG. 10A) AsCas12a V3 from IDT; FIG. 10B) AsCas12a Ultra from IDT; FIG. 10C) LbCas12a Ultra from IDT; FIG. 10D) EnGen Lba Cas12a from NEB. Due to the immediate reach of fluorescence saturation levels for high target concentrations, the fluorescence enhancement rates were estimated as the transient rates at the first 30 s of reaction. The inset figures are zoomed-in views when target concentrations were low. The points represent individual measurements, and the error bars represent means±standard deviation (s.d.) from three technical replicates. The unpaired two-sample t-test was used to analyze the statistical significance. *: p<0.05. ***: p<0.001. ***: p<0.0001. ns: not significant. The sensitivity was determined as the lowest target concentration with a fluorescence enhancement rate statistically significant to that of the blank control.

FIGS. 11A-D show fluorescence enhancement rates of ssDNA-activated trans-cleavage on ssDNA reporters with Cas12a enzymes: FIG. 11A) AsCas12a V3 from IDT; FIG. 11B) AsCas12a Ultra from IDT; FIG. 11C) LbCas12a Ultra from IDT; FIG. 11D) EnGen Lba Cas12a from NEB. Due to the immediate reach of fluorescence saturation levels for high target concentrations, the fluorescence enhancement rates were estimated as the transient rates at the first 30 s of reaction. The inset figures are zoomed-in views when target concentrations were low. The points represent individual measurements, and the error bars represent means±standard deviation (s.d.) from three technical replicates. The unpaired two-sample t-test was used to analyze the statistical significance. **: p<0.01. ***: p<0.001. ****: p<0.0001. ns: not significant. The sensitivity was determined as the lowest target concentration with a fluorescence enhancement rate statistically significant to that of the blank control.

FIGS. 12A-D shows fluorescence enhancement rates of ssDNA-activated trans-cleavage on ssRNA reporters with Cas12a enzymes: FIG. 12A) AsCas12a V3 from IDT; FIG. 12B) AsCas12a Ultra from IDT; FIG. 12C) LbCas12a Ultra from IDT; FIG. 12D) EnGen Lba Cas12a from NEB. The fluorescence enhancement rates were estimated as the average rates during the first 30 min of reaction. The inset figures are zoomed-in views when target concentrations were low. The points represent individual measurements, and the error bars represent means±standard deviation (s.d.) from three technical replicates. The unpaired two-sample t-test was used to analyze the statistical significance. *: p<0.05. **: p<0.01. ****: p<0.0001. ns: not significant. The sensitivity was determined as the lowest target concentration with a fluorescence enhancement rate statistically significant to that of the blank control.

FIGS. 13A-D shows fluorescence enhancement rates of ssRNA-activated trans-cleavage on ssRNA reporters with Cas12a enzymes: FIG. 13A) AsCas12a V3 from IDT; FIG. 13B) AsCas12a Ultra from IDT; FIG. 13C) LbCas12a Ultra from IDT; FIG. 13D) EnGen Lba Cas12a from NEB. The fluorescence enhancement rates were estimated as the average rates during the first 30 min of reaction. The inset figures are zoomed-in views when target concentrations were low. The points represent individual measurements, and the error bars represent means±standard deviation (s.d.) from three technical replicates. The unpaired two-sample t-test was used to analyze the statistical significance. **: p<0.01. ***: p<0.001. ****: p<0.0001. ns: not significant. The sensitivity was determined as the lowest target concentration with a fluorescence enhancement rate statistically significant to that of the blank control.

FIGS. 14A-C shows experimental results on the cis- and trans-activity of Cas12a with extended ssRNA activators. FIG. 14A) Comparison of real-time trans-cleavage fluorescence kinetics between 5′-extended ssRNA activator and both 5′- and 3′-extended ssRNA activator with AsCas12a Ultra (IDT). FIG. 14B) Comparison of real-time trans-cleavage fluorescence kinetics between 5′-extended ssRNA activator and both 5′- and 3′-extended ssRNA activator with LbCas12a Ultra (IDT). For A) and B), the experiments for each group were performed in triplicate and graphs represent means (bold line)+standard deviation (s.d.). FIG. 14C) Denaturing PAGE gel electrophoresis for identification of cis-cleavage and trans-cleavage products using 3′ FAM-labeled ssRNA target with both 5′ and 3′ extensions. For uncropped gel scan see FIG. 17.

FIG. 15 shows denaturing PAGE gel electrophoresis for identification of cis-cleavage products using ssDNA target with 5′ extensions. SYBR gold staining was applied to the gel to visualize the bands. For uncropped gel scan see FIG. 28.

FIGS. 16A-E shows a specificity comparison of trans-cleavage activity with Cas12a by ssDNA and ssRNA targets with single-base mutations. FIG. 16A) Schematic illustration of ssDNA- and ssRNA-activated trans-cleavage activity of Cas12a and the mutated target sequences used to investigate the specificity of trans-cleavage activity with ssDNA and ssRNA targets. Wild-type (WT) sequences refer to target sequences fully complementary to the spacer of crRNA. Mutation positions are marked in pale grey. FIGS. 16B-E) Measurements were carried out with FIG. 16B) AsCas12a V3 (IDT), FIG. 16C) AsCas12a Ultra (IDT), FIG. 16D) LbCas12a Ultra (IDT), and FIG. 16E) EnGen Lba Cas12a (NEB) by calculating the relative fluorescence enhancement rate compared to that for the fully matched target. The points represent individual measurements and the error bars represent means±standard deviation (s.d.) from three technical replicates. The experimental groups with negative fluorescence enhancement rates, indicating no reactivity to a mismatched target, were left blank in the figure.

FIGS. 17A-E shows the contribution of DNA to signal generation in the UNIVERSE assay by DNase-based amplicon removal. A-B) Real-time fluorescence curves showing the changes in trans-cleavage fluorescence signal with or without DNase treatment to the T7 transcription products. The CRISPR reactions were performed using FIG. 17A) AsCas12a Ultra (IDT) and FIG. 17B) LbCas12a Ultra (IDT). FIG. 17C) The real-time background fluorescence generated from DNase degrading the ssDNA reporters. The experiment was performed in triplicate and graphs represent means (bold line)±standard deviation (s.d.). D-E) The fluorescence enhancements over 120 min with FIG. 17D) AsCas12a Ultra (IDT) and E) LbCas12a Ultra (IDT) after subtraction of background fluorescence when the reaction was treated with DNase. Two concentrations of HIV p24 plasmid, 3×10⁶ copies/μL and 3×10⁴ copies/μL, are shown. The points represent individual measurements, and the error bars represent means±standard deviation (s.d.) from three technical replicates.

FIGS. 18A-D shows PAM-free detection of HIV plasmid using the UNIVERSE assay with AsCas12a enzymes. FIG. 18A) Real-time trans-cleavage fluorescence kinetics for detection with AsCas12a V3 (IDT) in the UNIVERSE assay. FIG. 18B) Real-time trans-cleavage fluorescence kinetics for detection with AsCas12a Ultra (IDT) in the UNIVERSE assay. FIG. 18C) End-point fluorescence enhancement at 120 min for detection with AsCas12a V3 (IDT) in the UNIVERSE assay. FIG. 18D) End-point fluorescence enhancement at 120 min for detection with AsCas12a Ultra (IDT) in the UNIVERSE assay. The detection was performed on 10-fold serially diluted HIV p24 plasmid starting at 3×10⁶ copies/μL. The inset figures are zoomed-in views when template concentrations were low. The points represent individual measurements and the error bars represent means±standard deviation (s.d.) from four technical replicates. The unpaired two-sample t-test was used to analyze the statistical significance. **: p<0.01. ****: p<0.0001. ns: not significant.

FIGS. 19A-D shows PAM-free detection of HIV plasmid using the conventional RPA/CRISPR-Cas12a assay with AsCas12a enzymes. FIG. 19A) Real-time trans-cleavage fluorescence kinetics for detection with AsCas12a V3 (IDT) in the conventional RPA/CRISPR-Cas12a assay. FIG. 19B) Real-time trans-cleavage fluorescence kinetics for detection with AsCas12a Ultra (IDT) in the conventional RPA/CRISPR-Cas12a assay. FIG. 19C) End-point fluorescence enhancement at 120 min for detection with AsCas12a V3 (IDT) in the conventional RPA/CRISPR-Cas12a assay. FIG. 19D) End-point fluorescence enhancement at 120 min for detection with AsCas12a Ultra (IDT) in the conventional RPA/CRISPR-Cas12a assay. The detection was performed on 10-fold serially diluted HIV p24 plasmid starting at 3×10⁶ copies/μL. The inset figures are zoomed-in views when template concentrations were low. The points represent individual measurements and the error bars represent means±standard deviation (s.d.) from four technical replicates. The unpaired two-sample t-test was used to analyze the statistical significance. ***: p<0.001. ****: p<0.0001. ns: not significant

FIGS. 20A-C shows data regarding the source of background signal in the UNIVERSE assay. FIG. 20A) Real-time fluorescence kinetics of the CRISPR trans-cleavage assay on forward and reverse primers used in the HIV p24 plasmid detection. FIG. 20B) Real-time fluorescence kinetics of the UNIVERSE assay on products of RPA reactions where both primers, a single primer, or no primers were added. The blank control without p24 plasmid template was applied to all RPA reactions. The purple curve represents a control where RPA products with both primers added were directly applied to a CRISPR assay without T7 RNAP to investigate the possibility of contamination. FIG. 20C) End-point fluorescence enhancement at 120 min for all reaction conditions in B). The points represent individual measurements, and the error bars represent means±standard deviation (s.d.) from four technical replicates.

FIGS. 21A-C shows a comparison of HIV plasmid detection between the conventional RPA/CRISPR-Cas12a assay detecting a PAM target and the UNIVERSE assay detecting a PAM-less target. FIG. 21A) Real-time trans-cleavage fluorescence kinetics for detection with LbCas12a Ultra (IDT) in the conventional RPA/CRISPR-Cas12a assay detecting a PAM target. FIG. 21B) End-point fluorescence enhancement at 120 min for detection with LbCas12a Ultra (IDT) in the conventional RPA/CRISPR-Cas12a assay detecting a PAM target. FIG. 21C) Comparison of average fluorescence enhancement rates between the conventional RPA/CRISPR-Cas12a assay detecting a PAM target and the UNIVERSE assay detecting a PAM-less target. To facilitate the comparison, the intensity of the trans-cleavage activity was represented by the logarithm of the average fluorescence enhancement rate from three technical replicates to base 10. The detection was performed on 10-fold serially diluted HIV p24 plasmid starting at 3×10⁶ copies/μL. The inset figure in B) is a zoomed-in view when the template concentrations were low. The points represent individual measurements and the error bars represent means±standard deviation (s.d.) from three technical replicates. The unpaired two-sample t-test was used to analyze the statistical significance. ***: p<0.001. ****: p<0.0001. ns: not significant.

FIG. 22 shows a flowchart of a program calculating the homology of a 20-nt target to be used in the CRISPR reaction. The complete algorithm is divided into a primer mismatch screening section and a CRISPR target mismatch screening section. In this exemplary logic diagram/logic program, a satisfying primer which is not assumed to inhibit the RPA reaction should contain at most one base of permutation, insertion or deletion excluding the first two bases at the 3′ end. As a more stringent CRISPR target selection criteria, the CRISPR target in a candidate amplicon to be checked should be identical to the CRISPR target in the reference amplicon. “Count_A” refers to the total number of candidate amplicons that meet the requirements of primer mismatch screening. “Count_B” refers to the total number of candidate amplicons containing a target matching the reference target. The match ratio is therefore “Count_B” divided by “Count_A.”

FIG. 23 shows quantification of copy numbers of 2-fold serially diluted HIV RNA-positive control by digital RT-PCR. The pale grey line indicates the linear fitting between the measured copy number and the supposed relative concentration to the undiluted sample. The fitting statistics are given in the inset table.

FIG. 24 shows microscopic fluorescence images of digital RT-PCR chips with amplifications of 2-fold serially diluted HIV RNA-positive control and nucleic acid extractions from healthy human plasma (NC). The dilution ratio is marked in each subfigure.

FIGS. 25A-B shows real-time fluorescence kinetics for clinical sample detection. FIG. 25A) Detection of HIV clinical samples using the UNIVERSE assay. FIG. 25B) Detection of HPV 16 clinical samples using the UNIVERSE assay.

FIG. 26 shows uncropped gel image of FIG. 1F. The black-boxed region in the uncropped gel image was used.

FIG. 27 shows uncropped gel image of FIG. 14C. The black-boxed region in the uncropped gel image was used.

FIG. 28 shows uncropped gel image of FIG. 15. The black-boxed region in the uncropped gel image was used.

FIG. 29 shows uncropped gel image FIG. 3B. The black-boxed region in the uncropped gel image was used. The ss20 DNA ladder (10-200 nt) obtained from Simplex Sciences (New Haven, CT) was used as the size marker.

### DETAILED DESCRIPTION

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used herein the terms “disease,” “disease state” and “pathologic condition” are used interchangeably, unless indicated otherwise herein, to describe a deviation from the condition regarded as normal or average for members of a species or group (e.g., humans), and which is detrimental to an affected individual under conditions that are not inimical to the majority of individuals of that species or group. Such a deviation can manifest as a state, signs, and/or symptoms that are associated with any impairment of the normal state of a subject or of any of its organs or tissues that interrupts or modifies the performance of normal functions. A disease or pathological condition may be caused by or result from contact with a pathogenic microbe, may be responsive to environmental factors (e.g., malnutrition and/or industrial hazards), may be responsive to an inherent or latent defect in the organism (e.g., genetic anomalies) or to combinations of these and other factors.

The terms “host,” “subject,” or “patient” are used interchangeably herein and include, but are not limited to, mammals (e.g., murines, simians, equines, bovines, porcines, canines, felines, and the like), and most preferably includes humans. In the context of the disclosure, the term “subject” generally refers to an individual from whom a sample is obtained according to the methods disclosed herein.

The term “solution” refers to an aqueous or non-aqueous mixture.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Where particular values are described in the application and claims, unless otherwise stated, the term “about” is implicit and in this context means within an acceptable error range for the particular value.

Conventional Cas12a assays (e.g., the DETECTR assay, Mammoth Biosciences) depend on a PAM (protospacer adjacent motif) near the target site to enable strand separation and crRNA binding. This requirement limits target selection because only sequences adjacent to a suitable protospacer adjacent motif (PAM) can be recognized and cleaved. Additionally, when activated by DNA, Cas12a leads to cleavage of the target sequence itself (i.e., cis-cleavage), reducing the overall sensitivity of the detection assay.

Experiments conducted during development of embodiments of the present disclosure identified for the first time that Cas12a can be directly activated by a complementary RNA target (e.g., ssRNA target), triggering robust trans-cleavage activity of Cas12a. Moreover, as described in the Examples, RNA activation of Cas12a yields improved specificity, in particular its ability to discriminate sequences comprising one or more mutations, compared to traditional Cas12a DNA activation (e.g., the DETECTR assay). Moreover, as detailed herein, a nucleic acid detection assay and systems comprising same of the disclosure comprise a CRISPR-Cas12a protein and a T7 transcription step that converts nucleic acid targets lacking a protospacer adjacent motif (PAM) into single stranded RNA (ssRNA) targets (e.g., thereby circumventing the requirement of conventional CRISPR assays and systems that require target substrates contain a PAM sequence). Assays disclosed in the present disclosure (e.g., the UNIVERSE assay) exhibit enhanced sensitivity and signal strength (e.g., for the detection of clinical sample nucleic acids such as, but not limited to, pathogenic microbes such as HIV and HPV) and outperform conventional RPA/CRISPR-Cas12a assays (e.g., the DETECTR assay).

As detailed herein, by incorporating a T7 transcription step in conventional CRISPR-based assays (e.g., conventional assays such as DETECTR), the new assay of the present disclosure directly detects full-length RNA by CRISPR-Cas12a and is capable of detecting random targets, thereby overcoming the difficulty of using conventional CRISPR-based assays to detect highly homologous sequences without a protospacer adjacent motif (PAM).

In an embodiment, the UNIVERSE assay achieves random target selection for detection using Cas12a, which enables it to detect highly consensus target sequences in virus genome and enhances the reliability in detection. The UNIVERSE assay achieves similar sensitivity in nucleic acid detection reaching about 3 copies/μL, akin to the high sensitivity observed using qPCR detection (e.g., and about 10-100-fold more sensitive than conventional assays). By detecting RNA instead of DNA, the UNIVERSE assay also achieves significantly higher specificity in detecting mutated nucleic acid sequences, thus providing more accuracy in nucleic acid testing.

As detailed herein and described in the Examples, Cas12a orthologs were discovered to be directly activated by a fully complementary RNA target without the need for additional DNA activators. Noticeable differences in cis-activity were also discovered (See Examples), wherein the DNA target was prone to degradation while the RNA target exhibited significant resistance. In addition, the specificity towards RNA targets was greatly improved compared to DNA targets.

The UNIVERSE assay provided by the disclosure is a universal nucleic acid detection method/assay that, in some embodiments, possesses improved sensitivity and specificity, for example, by incorporating T7 transcription to convert dsDNA amplicons to ssRNA. This unique detection modality eliminates the need to search for a canonical PAM or a suboptimal PAM sequence, or to resort to the complicated experimental design of artificially introducing a PAM.

The broad, clinical utility of the UNIVERSE assay disclosed herein was demonstrated by detecting multiple pathogenic microbes including HIV in clinical plasma samples and HPV 16 in clinical cervical swab samples (See Examples). Detection/diagnostic performance was similar to that of RT-PCR/PCR methodology. The UNIVERSE assay can be conducted at steady temperatures making it superior to the RT-PCR/PCR method in terms of application in resource-limited settings (e.g., where a thermocycler is unavailable). Compared to the Cas13a system, a prevalent technique in nucleic acid detection that utilizes a similar trans-cleavage mechanism upon RNA targeting, the UNIVERSE assay with Cas12a can be accomplished with shorter RNA guides and more durable ssDNA probes/reporters. As the synthesis of RNA oligos is expensive, the disclosure provides realization of reduced overall cost for detection/diagnosis compared to conventional assays. In some embodiments, compositions and methods of the disclosure are integrated with microfluidics technology making UNIVERSE a valid point-of-care testing tool.

Sensitivity. The UNIVERSE assay detects nucleic acids at much lower template concentrations than conventional Cas12a assays. For example, as shown in the Examples, when using LbCas12a Ultra, the UNIVERSE assay reached a detection limit of about 3 copies/μL, whereas the conventional RPA/CRISPR-Cas12a assay required around 300 copies/μL—a roughly 100-fold improvement. Similarly, with the AsCas12a enzymes, the detection limit improved from about 300 copies/μL in the conventional assay to around 30 copies/μL in the UNIVERSE assay, a 10-fold enhancement.

Specificity. As detailed herein, RNA-activation markedly improved target discrimination compared to conventional DNA-activation. For example, as shown in the Examples, when using RNA targets, most mismatched sequences produced significantly lower fluorescence signals relative to the fully matched target. In contrast, with DNA activation, several mismatched targets-even those with two-base mismatches-sometimes yielded fluorescence signals equal to or even exceeding those of the perfectly matched target. Accordingly, compositions and methods of the disclosure that utilize RNA activation reduced false-positive signals and improved mismatch discrimination significantly (in some cases, effectively eliminating problematic signals seen with DNA targets). RNA-activated Cas12a is better at reliably distinguishing mutated targets from correct ones, providing a more accurate diagnostic than conventional assays.

Versatility. The UNIVERSE assay offers several examples of enhanced versatility over conventional Cas12a assays. The methods of the disclosure overcome the PAM requirement by incorporating a T7 transcription step. This means that any region within an amplicon-regardless of adjacent PAM sequences—can be targeted, allowing for the random selection of target sequences.

Reduced overall cost and increased stability. The UNIVERSE assay reduces costs in several practical ways. In some embodiments, it uses shorter RNA guides compared to alternative CRISPR systems (like Cas13a), which are both less expensive to synthesize and less prone to degradation. This means fewer losses over time and less need for stringent storage conditions. The disclosure also reduces costs eliminating the need for designing and incorporating additional PAM sequences or specialized primers resulting in a simplified workflow. This streamlined design reduces reagent complexity and associated costs. Furthermore, the increased sensitivity of the UNIVERSE assay means that lower amounts of sample and reagents are required to achieve reliable detection. This improved efficiency can reduce the per-test reagent volume and overall cost. Use of ssDNA reporters, which are less expensive and more stable than longer RNA counterparts, further contributes to cost savings. Thus, the disclosure provides not only lower material expenses but also reduced operational costs (e.g., by providing, in some embodiments, a robust, single-vessel/tube reaction with minimal reagent overhead).

Increased reliability. The UNIVERSE assay enhances reliability through several key features. UNIVERSE eliminates the PAM constraint by converting dsDNA amplicons into ssRNA via T7 transcription. This means that the assay is not limited by variable or suboptimal PAM sequences, ensuring consistent activation of Cas12a across different target regions. Furthermore, as detailed herein, RNA-activated Cas12a exhibits minimal cis-cleavage of the target molecule. By preserving the integrity of the RNA target, the assay maintains a robust signal and avoids target degradation that can compromise detection reliability. Improved specificity of RNA activation reduced false-positive signals. When mismatches occur, the RNA-triggered trans-cleavage response is sharply diminished compared to DNA activation, leading to more accurate discrimination between true and false signals. Thus, the UNIVERSE assay delivers more consistent, accurate, and robust diagnostic performance compared to conventional Cas12a-based assays.

The UNIVERSE assay can be used in a broad range of diagnostic tests, for example, as described herein. For instance, by bypassing the need for a protospacer adjacent motif (PAM), the assay can target any region within a nucleic acid amplicon. This flexibility is particularly useful for emerging pathogens or cases where the target region does not contain a canonical PAM. Moreover, the assay's streamlined format that, in some embodiments, integrates amplification, T7 transcription, and CRISPR detection (e.g., all in one reaction vessel or tube) can be adapted into rapid, field-deployable diagnostic platforms, making it valuable in resource-limited settings. Furthermore, in some embodiments, the UNIVERSE assay can be multiplexed (e.g., parallel detection of multiple microbes) and used in broad-spectrum diagnostics. For example, UNIVERSE assay works with both DNA and RNA targets which makes it a versatile tool for designing multiplex panels that can simultaneously detect a range of pathogens or genetic markers, (e.g., for use during infectious disease outbreaks or for comprehensive screening programs).

Embodiments of the UNIVERSE assay described herein provide a robust CRISPR-Cas12a-based diagnostic with attomolar sensitivity. For example, the embodiments of the UNIVERSE assay described herein provide a robust CRISPR-Cas12a-based diagnostic with about 1-5 attomolar sensitivity, about 1-4 attomolar sensitivity, about 2-4 attomolar sensitivity, or about 3-4 attomolar sensitivity. In some embodiments, the UNIVERSE assay described herein provides a robust CRISPR-Cas12a-based diagnostic with about 3-3.5 attomolar sensitivity. Embodiments disclosed herein can detect DNA and RNA with comparable levels of sensitivity and can differentiate targets from non-targets based on single base pair differences. Moreover, the embodiments disclosed herein can be prepared in a lyophilized or freeze-dried format for convenient distribution and point-of-care (POC) applications.

In one embodiment, the disclosure provides a method for detecting a microbe and/or microbial nucleic acid in a sample comprising obtaining a sample suspected of containing microbial nucleic acid from a subject; generating amplicons of the microbial nucleic acid via contacting the sample suspected of containing microbial nucleic acid with recombinase polymerase and reagents for amplification of the microbial nucleic acid and amplifying the microbial nucleic acid via recombinase polymerase amplification (RPA), wherein the amplicons of the microbial nucleic acid lack protospacer adjacent motif (PAM) sequences; transcribing the amplicons of the microbial nucleic acid into single-stranded RNA (ssRNA) targets using T7 RNA polymerase; incubating the ssRNA targets with a CRISPR-Cas12a nuclease, one or more guide RNA sequences specific for one or more regions within the ssRNA targets, and reporter molecules (e.g., under conditions sufficient to allow binding of the one or more guide RNA sequences to one or more regions within the ssRNA targets); activating the CRISPR-Cas12a nuclease via binding of the one or more guide RNA sequences to the one or more regions within the ssRNA targets; and generating a detectable signal via CRISPR-Cas12a trans-cleavage of the reporter molecules. In some embodiments, the presence of a detectable signal indicates the presence of the microbial nucleic acid in the sample. In some embodiments, the subject is a mammal such as human, cattle, cow, dog, cat, or pig. In some embodiments, the subject is human. In some embodiments, the absence of detectable signal indicates the absence of the microbial nucleic acid in the sample.

In some embodiments, each of the steps of a method for detecting a microbe and/or microbial nucleic acid in a sample described herein occur in the same tube and/or vessel. In some embodiments, nucleic acid is extracted and/or purified from the sample (e.g., prior to generating amplicons of the microbial nucleic acid). In some embodiments, generating amplicons of the microbial nucleic acid comprises use of a primer comprising a T7 promoter sequence (e.g., a sequence comprising SEQ ID NO. 11, SEQ ID NO. 16, SEQ ID NO. 24, or SEQ ID NO. 33) and other reagents for amplification of microbial nucleic acid. Other reagents for amplification of microbial nucleic acid comprise amplification reagents that are generally known in the art. For example, an amplification reagent as described herein may include a buffer, such as a Tris buffer. A Tris buffer may be used at any concentration appropriate for the desired application or use as described herein, for example, and include but are not limited to a concentration of about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present disclosure.

A salt, such as magnesium chloride (MgCl2), potassium chloride (KCl), or sodium chloride (NaCl), may be included in an amplification reaction in order to improve the amplification of nucleic acid. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present disclosure and as described herein. Other components may include a cell lysis component in order to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be used include Triton X-100, sodium dodecyl sulfate (SDS), CHAPS (3-[(3-cholamidopropyl) dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate as disclosed herein, such as including, but not limited to, a concentration of about 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like.

In some embodiments, microbial nucleic acid detected by a method for detecting a microbe and/or microbial nucleic acid in a sample described herein is DNA. In other embodiments, the microbial nucleic acid is RNA. In some embodiments, microbial nucleic acid comprises DNA and RNA. In some embodiments, the UNIVERSE assay comprises generating amplicons of the microbial nucleic acid comprising amplifying the microbial nucleic acid via recombinase polymerase amplification (RPA), wherein the amplicons of the microbial nucleic acid lack protospacer adjacent motif (PAM) sequences. In addition to recombinase polymerase amplification (RPA), other isothermal amplification may be used including, but not limited to, nucleic-acid sequenced-based amplification (NASBA), loop-mediated isothermal amplification (LAMP), strand displacement amplification (SDA), helicase-dependent amplification (HDA), and/or nicking enzyme amplification reaction (NEAR).

In some embodiments, activating the CRISPR-Cas12a nuclease occurs via binding of the one or more guide RNA sequences to the one or more regions within the ssRNA targets. The disclosure is not limited by the guide RNA sequence utilized. Indeed, any guide RNA sequence may be used to target Cas12a nuclease to ssRNA targets generated via T7 RNA polymerase transcription of amplicons of the microbial nucleic acid lacking protospacer adjacent motif (PAM) sequences. Exemplary guide RNA sequences include but are not limited to SEQ ID NO. 13, SEQ ID NO. 18, SEQ ID NO. 26, SEQ ID NO. 35, SEQ ID NO. 36, SEQ ID NO. 37, SEQ ID NO. 38, SEQ ID NO. 39, and SEQ ID NO. 40. A “guide sequence,” “crRNA,” “guide RNA,” or “guide RNA sequence” refers to a polynucleotide sequence having sufficient complementarity with a ssRNA target (e.g., ssRNA target generated via T7 RNA polymerase transcription of amplicons of the microbial nucleic acid lacking protospacer adjacent motif (PAM) sequences) to hybridize with the ssRNA target nucleic acid sequence and to direct sequence-specific binding of a CRISPR-Cas12a complex comprising the guide sequence and CRISPR-Cas12a to the ssRNA target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences known in the art. The ability of a guide sequence to direct sequence-specific binding of a CRISPR-Cas12a complex comprising the guide sequence and CRISPR-Cas12a to a target nucleic acid sequence may be assessed by any suitable assay, including those described herein (e.g., in the Examples). In some embodiments, a guide RNA sequence is about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, the guide sequence is about 20 to 40 nucleotides long (e.g., 20, 21, 22, 23, 24, 25, 26, 27, 28, 29, 30, 31, 32, 33, 34, 34, 35, 36, 37, 38, 39 or 40 nucleotides long).

The method is not limited by the type of sample used in the methods for detecting a microbe and/or microbial nucleic acid in a sample described herein. Indeed, a variety of samples may be used including but not limited to blood, mucous, serum, plasma, saliva, urine, stool, vaginal fluid, synovial fluid, spinal fluid, and/or semen. In some embodiments, the sample is an upper respiratory sample (e.g., obtained from a nasopharyngeal swab, oropharyngeal (throat) swab, mid-turbinate nasal swab, anterior nasal swab, nasopharyngeal wash/aspirate and/or nasal aspirate).

The method is not limited by the reporter molecule used. A variety of reporter molecules are known in the art and can be used in methods disclosed herein including but not limited to fluorophore-quencher reporters (e.g., single-stranded DNA (ssDNA) sequence tagged with a fluorophore and quencher and/or a ssRNA sequence tagged with a fluorophore and quencher). For example, in some embodiments, the reporter molecule comprises SEQ ID NO. 5 and/or SEQ ID NO. 6. A “reporter molecule” refers to a molecule that can be cleaved or otherwise modified (e.g., activated or deactivated) by an activated CRISPR-Cas12a nuclease described herein. Other reporter systems known in the art (e.g., a colorimetric reporter system, a chemiluminescent reporter system, other fluorescent reporter systems, and/or any other detectable signal system) may be used with the assays of the present disclosure). In some embodiments, a reporter system is immobilized on a solid substrate (e.g., for use in microfluidics based detection).

The method is not limited to the type of microbial nucleic acid detected. For example, microbial nucleic acid may be from any one or more pathogenic viruses, bacteria, protozoa, and/or fungi described herein. In some embodiments, methods for detecting a microbe and/or microbial nucleic acid in a sample is used to diagnose and/or detect the presence of an infection and/or infectious agent in the sample and/or in the subject from which the sample was obtained. The disclosure is not limited by the type of infectious agent and/or infection detected using the compositions, methods and systems described herein. In some embodiments, the infection is a viral infection. In some embodiments, the infection is a bacterial infection. In other embodiments, the infection is a fungal infection. In some embodiments, the infection is a yeast infection. Examples of viral infections include but are not limited to infections caused by a Double-Stranded DNA virus (e.g., Herpesviridae including herpes simplex viruses (HSV-1 and HSV-2), varicella-zoster virus (VZV), and Epstein-Barr virus (EBV); Adenoviridae; Poxviridae such as variola virus and vaccinia virus; Papillomaviridae including but not limited to human papillomaviruses (HPV); and Hepadnaviridae such as hepatitis B virus (HBV)); infections caused by a Single-Stranded DNA Virus (e.g., Parvoviridae such as parvovirus B19); infections caused by a Positive-Sense Single-Stranded RNA Virus (e.g., Picornaviridae including poliovirus and rhinoviruses, Flaviviridae that include dengue virus, Zika virus, West Nile virus, and yellow fever virus. Togaviridae such as the chikungunya virus, and Coronaviridae that include SARS-CoV, MERS-COV, and SARS-COV-2); infections caused by a Negative-Sense Single-Stranded RNA Virus (e.g., Orthomyxoviridae such as influenza viruses. Paramyxoviridae including measles and mumps viruses, and respiratory syncytial virus (RSV), Rhabdoviridae such as rabies virus. Filoviridae including Ebola and Marburg viruses, and Arenaviridae and Bunyaviridae that include viruses causing hemorrhagic fevers and encephalitis); infections caused by a Double-Stranded RNA Virus (e.g., Reoviridae including rotaviruses); and/or infections caused by a Retrovirus such as human immunodeficiency virus (HIV). Examples of bacterial infections include but are not limited to infections caused by a Gram-Positive Bacteria (e.g., Staphylococcus aureus, Streptococcus pyogenes (Group A Streptococcus), Streptococcus pneumoniae, Bacillus anthracis: Clostridium species including Clostridium botulinum, Clostridium tetani, and Clostridium difficile, and Listeria monocytogenes), Gram-Negative Bacteria (e.g., Neisseria gonorrhoeae, Neisseria meningitidis, Escherichia coli, Salmonella enterica, Shigella species, Pseudomonas aeruginosa, and Klebsiella pneumoniae), and Atypical Bacteria (e.g., Mycobacteria such as Mycobacterium tuberculosis and Mycobacterium leprae, Spirochetes such as Treponema pallidum and Borrelia burgdorferi, Chlamydia trachomatis, and Rickettsia species). Examples of fungal infections include but are not limited to infections caused by Aspergilli, Candidae, Candida albicans, Coccidioides immitis, Cryptococci, and/or combinations thereof. Examples of yeast infections include but are not limited to infections caused by Candida albicans, Candida glabrata, Candida tropicalis, Candida parapsilosis, Candida krusei, Cryptococcus neoformans, and/or Cryptococcus gattii. The method may be used for the detection of nucleic acid from a single microbe, or it may be used for the parallel, simultaneous detection of nucleic acid (e.g., ssRNA targets generated according to the method) from a plurality of different pathogenic microbes. Indeed, the method may be used to detect nucleic acid (e.g., ssRNA targets generated according to the method) of a plurality of different viruses, bacteria, protozoa, and/or fungi. In some embodiments, the activated CRISPR-Cas12a nuclease does not cleave the ssRNA targets. The method may be utilized in a variety of ways and settings. For example, in some embodiments, the method is used in a lateral flow immunochromatographic assay. In some embodiments, the method is performed more than one time from different samples obtained from the same subject over a period of time. The disclosed methods may be used to distinguish between two or more species of one or more organisms in a sample, or, alternatively, to detecting one or more species of one or more organisms in the sample. In some embodiments, the UNIVERSE assay is used to detect resistance of a microbe to one or more antibiotics (e.g., via detection of nucleic acid sequence in a antibiotic and/or viral resistance gene of a microbe). In some embodiments, the UNIVERSE assay is used to detect outbreaks and/or monitor community progression of infectious disease (e.g., via detection of microbial nucleic acid of a microbe responsible for the infectious disease outbreak). In other embodiments, the UNIVERSE assay is used to detect environmental microbial contamination (e.g., food contamination by pathogenic bacteria).

In some embodiments, the UNIVERSE assay is used to detect the presence of cancer, neoplasm and/or tumor in a subject. The disclosure is not limited by the type of cancer, tumor and/or neoplasm detected. Examples of general categories of cancer detected and/or diagnosed include but are not limited to carcinomas (i.e., malignant tumors derived from epithelial cells such as, for example, common forms of breast, prostate, lung and colon cancer), sarcomas (i.e., malignant tumors derived from connective tissue or mesenchymal cells), lymphomas (i.e., malignancies derived from hematopoietic cells), leukemias (i.e., malignancies derived from hematopoietic cells), and germ cell tumors (i.e., tumors derived from totipotent cells. Examples neoplasms and/or tumors detected and/or diagnosed include but are not limited to those neoplasms associated with cancers of neural tissue, blood forming tissue, breast, skin, bone, prostate, ovaries, uterus, cervix, liver, lung, brain, larynx, gallbladder, pancreas, rectum, parathyroid, thyroid, adrenal gland, immune system, head and neck, colon, stomach, bronchi, and/or kidneys. In some embodiments, the UNIVERSE assay detects, diagnoses, and/or identifies a disease and/or disease state in a subject via detection of target nucleic acids comprising a mutation and/or polymorphism. In some embodiments, the mutation is a single nucleotide change such as a point mutation (e.g., a missense mutation, a nonsense mutation, a silent mutation, a single nucleotide polymorphism (SNP). In other embodiments, the mutation and/or polymorphism is an insertion or deletion (Indel), a frameshift mutation or a splice variation. In still other embodiments, the mutation or polymorphism is an inversion or translocation. In some embodiments, the UNIVERSE assay detects, diagnoses, and/or identifies a disease and/or disease state in a subject via detection of target nucleic acids lacking a PAM sequence (e.g., the ability of the UNIVERSE assay to detect nucleic acid sequence lacking a protospacer adjacent motif (PAM) sequence makes possible detection of any region within a nucleic acid amplicon).

The UNIVERSE assay may be integrated into and/or used in conjunction with a diagnostic device known in the art. Such devices include, but are not limited to, a flow strip, a microfluidic device, a flexible material based substrate (See, e.g., International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719 (2015)), flow cytometer, a wearable medical device, a point of care device, a lab on chip sensing device, or other device known in the art (e.g., that permits sensing of reporter molecules).

The disclosure also provides a kit for detecting a microbial nucleic acid in a sample. In some embodiments, the kit comprises recombinase polymerase, a primer comprising a T7 promoter sequence and other reagents for amplification of the microbial nucleic acid (e.g., sufficient to generate amplicons of the microbial nucleic acid lacking protospacer adjacent motif (PAM) sequences), T7 RNA polymerase (e.g., for transcribing amplicons of the microbial nucleic acid into single-stranded RNA (ssRNA) targets), CRISPR-Cas12a nuclease; one or more guide RNA sequences (e.g., specific for one or more regions within the ssRNA targets), and reporter molecules. Descriptions of the primers comprising a T7 promoter sequence and other reagents for amplification of the microbial nucleic acid, T7 RNA polymerase, CRISPR-Cas12a nuclease, guide RNA sequences, and reporter molecules set forth herein with respect to the aforementioned methods and assays also are applicable to those same aspects of the kits described herein. Additional examples of suitable reagents for inclusion in the kit include conventional reagents employed in nucleic acid amplification reactions, such as, for example, one or more enzymes having polymerase activity, enzyme cofactors (such as magnesium or nicotinamide adenine dinucleotide (NAD)), salts, buffers, deoxyribonucleotide, or ribonucleotide triphosphates (dNTPs/rNTPs; for example, deoxyadenosine triphosphate, deoxyguanosine triphosphate, deoxycytidine triphosphate, and deoxythymidine triphosphate) blocking agents, labeling agents, and the like. Many such reagents are described herein or otherwise known in the art and commercially available. In an embodiment, the primer comprising a T7 promoter sequence comprises SEQ ID NO. 11, SEQ ID NO. 16, SEQ ID NO. 24, or SEQ ID NO. 33. In one embodiment, in addition to the conventional reagents employed in nucleic acid amplification reactions (e.g., for amplification of the microbial nucleic acid sufficient to generate amplicons of the microbial nucleic acid lacking protospacer adjacent motif (PAM) sequences), the kit also comprises a primer sequence comprising SEQ ID NO. 12, SEQ ID NO. 17, SEQ ID NO. 25, and/or SEQ ID NO. 34. In some embodiments, the one or more guide RNA sequences comprise SEQ ID NO. 13, SEQ ID NO. 18, SEQ ID NO. 26, SEQ ID NO. 35, SEQ ID NO. 36, SEQ ID NO. 37, SEQ ID NO. 38, SEQ ID NO. 39, or SEQ ID NO. 40. In some embodiments, reporter molecules comprise SEQ ID NO. 5 and/or SEQ ID NO. 6. In some embodiments, the kit comprises a control target sequence comprising SEQ ID NO. 14, SEQ ID NO. 19, SEQ ID NO. 27, SEQ ID NO. 28, or SEQ ID NO. 41.

The kit may comprise instructions for using the UNIVERSE assay described herein, e.g., for obtaining and/or processing the sample, optionally extracting nucleic acid molecules, and/or performing the UNIVERSE assay; and for interpreting the results obtained, as well as a notice in the form prescribed by a governmental agency. Such instructions optionally can be in printed form or on CD, DVD, or other format of recorded media.

The kits and/or composition may be supplied in a solid (e.g., lyophilized) or liquid form. The various components of the kits and composition of the present disclosure may optionally be contained within different containers (e.g., vessel, vial, ampoule, tube (e.g., test tube), flask, or bottle) for each individual component. Each component will generally be suitable as aliquoted in its respective container or provided in a concentrated form. Other containers suitable for conducting certain steps of the UNIVERSE assay may also be provided. The individual containers are preferably maintained in close confinement for commercial sale.

One of ordinary skill in the art, based on the present disclosure, can utilize the compositions and methods described to their fullest extent. The specific embodiments are therefore to be construed as merely illustrative, and not limitative of the remainder of the disclosure in any way. All publications cited herein are incorporated by reference for the purposes or subject matter referenced herein. From the above description, one skilled in the art can easily ascertain the essential characteristics of the present disclosure, and without departing from the spirit and scope thereof, can make various changes and modifications of the disclosure to adapt it to various usages and conditions.

### EXAMPLES

Examples of specific embodiments for carrying out the present disclosure are provided. The examples are offered for illustrative purposes only and are not intended to limit the scope of the present disclosure in any way.

### Example 1—Materials and Methods

Ethical statement. All clinical samples, including human plasma samples for HIV detection and cervical swab samples for HPV 16 detection, were de-identified and in compliance with ethical regulations and under the approval of the Institutional Review Board of the University of Connecticut Health Center.

Oligonucleotides used were obtained from Integrated DNA Technologies (IDT) and dissolved in nuclease-free water from New England Biolabs (NEB). A list of sequences can be found in Tables 1-7. The Cas12a enzymes, including ALT-R A.s. Cas12a (Cpf1) V3 (AsCas12a V3), ALT-R A.s. Cas12a (Cpf1) Ultra (AsCas12a Ultra), and ALT-R L.b. Cas12a (Cpf1) Ultra (LbCas12a Ultra), were purchased from IDT. The ENGEN Lba Cas12a enzyme was purchased from NEB. Prior to use, the Cas12a enzymes were diluted to 5 μM in 1×PBS (GIBCO, Thermo Fisher Scientific) and stored no longer than 24 h at 4° C. The buffer used in all CRISPR reactions was NEBuffer r3.1 from NEB. The TwistAmp Basic kit used for the RPA reactions was purchased from TwistDx Limited. For assembly of the UNIVERSE reaction, SuperScrip IV reverse transcriptase was obtained from Invitrogen. RNase H, RNAPol reaction buffer, ribonucleotide solution mix, RNase inhibitor (murine), and T7 RNA polymerase were obtained from NEB. For DNase removal of the RPA amplicon, DNase I-XT was obtained from NEB. For gel electrophoresis, 40% acrylamide/bis solution (19:1), 10×TBE buffer, ammonium persulfate (APS), and TEMED were purchased from Bio-Rad Laboratories. Urea (ACS reagent) was obtained from Sigma-Aldrich. ss20 DNA ladder was obtained from Simplex Sciences (New Haven, CT). The 2× Invitrogen Gel Loading Buffer II and SYBR Gold Nucleic Acid Gel Stain (10,000× Concentrate in DMSO) were obtained from Thermo Fisher Scientific. For viral nucleic acid extraction and quantitative PCR (qPCR) detection, AcroMetrix HIV High Control was obtained from Thermo Fisher Scientific. The QIAamp Viral RNA Mini Kit and DNeasy Blood and Tissue Kit were purchased from QIAGEN. The GoTaq Probe 1-Step RT-qPCR Kit was purchased from Promega. The 1× Reliance One-Step Multiplex Supermix for digital PCR was obtained from Bio-Rad Laboratories.

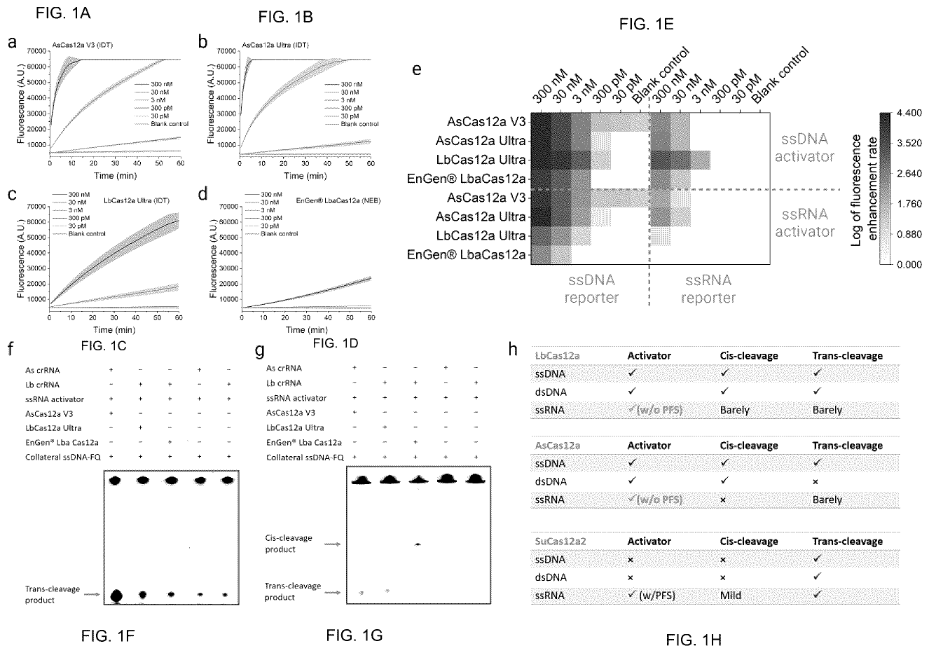

CRISPR trans-cleavage assay with single-stranded targets. For comparison of trans-cleavage performance under different conditions described in FIG. 1E, the 25 μL reaction was composed of 1× NEBuffer r3.1, 5 μM of ssDNA-FQ (/5′6-FAM/TTATT/3′IABKFQ/), 300 nM of crRNA, 300 nM of Cas12a enzyme, and 10-fold serially diluted single-stranded 20-nt ssDNA or ssRNA target where the highest concentration was 300 nM. For experiments with ssRNA reporters, the same concentration of ssRNA-FQ (/5′6-FAM/rUrUrUrUrU/3′IABKFQ/) was used. After thoroughly mixing the components and a brief centrifugation, the reaction was incubated at 37° C. in the CFX96 Touch Real-Time PCR Detection System (Bio-Rad Laboratories) for 1 h of fluorescence monitoring with a recording interval of 15 s.

Michaelis-Menten kinetics measurement. The Michaelis-Menten kinetics was measured following the protocol given by Ramachandran et al. Briefly, the experimental procedures were as follows: 1) 100 nM of Cas12a nuclease was incubated with 125 nM of crRNA in 1×NEBuffer r3.1 at 37° C. for 30 min. 2) The Cas12a RNP was mixed with a 10-fold molar excess of ssRNA activator. Specifically, 2 nM of Cas12a RNP was incubated with 20 nM of ssRNA activator in 1×NEBuffer r3.1 at 37° C. for 30 min. 3) The trans-cleavage assay was initiated by incubating 1 nM of the activated enzyme from Step 2 with 125 nM, 250 nM, 500 nM, 1 μM, 2 μM, and 4 μM of the ssDNA reporters in 1×NEBuffer r3.1 at 37° C. The fluorescence readouts were obtained every 15 s and the first 600 s of the fluorescence data were used to calculate the initial reaction velocities. The fluorescence data used in the calculation was a result of the raw fluorescence data background-subtracted by the average fluorescence intensities from four buffer-only samples.

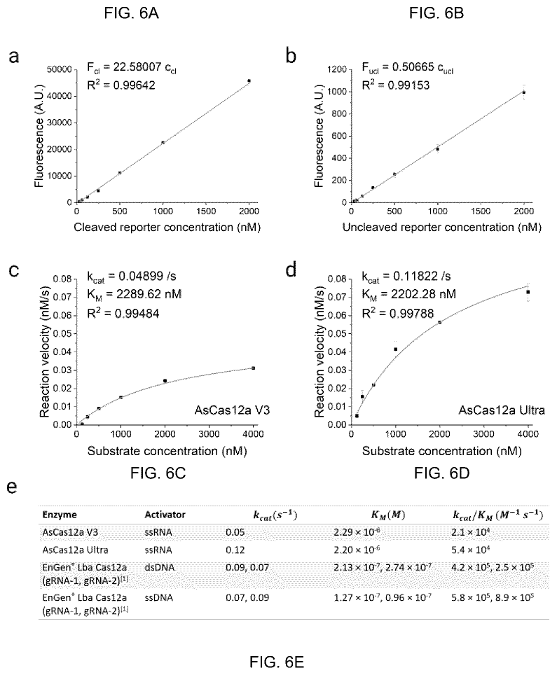

The calibration of fluorescence versus substrate concentration was carried out with concentrations of reporters varying from 31.25 nM to 2 μM, pre-cleaved in a reaction containing 1× NEBuffer r3.1, 300 nM of AsCas12a Ultra, 300 nM of crRNA, and 300 nM of ssRNA activator. The reaction lasted 10 h at 37° C., and at the end of the reaction the fluorescence values for all reporter concentrations were constant. A linear fitting between background-subtracted fluorescence Fel and cleaved reporter concentration c_{cl} was performed (FIG. 6A), given by F_{cl}=22.58007 c_{cl}, where c_{cl} is in a unit of nM and For is in the arbitrary fluorescence units (AU) of our thermal cycler. Similarly, the calibration of fluorescence versus uncleaved reporter concentration was performed (FIG. 6B). A linear fit was obtained between background-subtracted fluorescence F_{ucl} and uncleaved reporter concentration c_{ucl}, given by F_{ucl}=0.50665 c_{ucl}, where c_{ucl} is in a unit of nM and F_{ucl} is in the AU of our thermal cycler.

Next, a mathematical model was established to estimate the reaction velocity based on the calibration curves. Theoretically, the background-subtracted fluorescence F(t) as a function of time should be contributed by fluorescence from both the cleaved reporters F_{cl}(t) and uncleaved reporters F_{ucl} (t):

$$F ( t ) = {F}_{cl} ( t ) + {F}_{ucl} ( t ) \qquad ( 1 )$$

From the linear fitting results:

$$F ( t ) = 22.5 \; 8007 {c}_{cl} ( t ) + 0.5 \; 0665 {c}_{ucl} ( t ) \qquad ( 2 )$$

With c_{cl}(t)+c_{ucl} (t)=c₀, where c₀ is the initial concentration of uncleaved reporters, Eq. (2) can be transformed to:

$$F ( t ) = 22.5 \; 8007 {c}_{cl} ( t ) + 0.50665 ( {c}_{0} - {c}_{cl} ( t ) ) = 22.0 \; 7342 {c}_{cl} ( t ) + 0.5 \; 0665 {c}_{0} \qquad ( 3 )$$

The reaction velocity dc_{cl}/dt in nM/s is obtained by differentiating Eq. (3) with respect to time as:

$$\frac{{dc}_{cl}}{dt} = \frac{1}{22.07342} \times \frac{dF}{dt} \qquad ( 4 )$$

In this way, the reaction velocity can be calculated in nM/s by measuring the fluorescence enhancement rate in AU/s.

The reaction velocity was obtained from data for each substrate concentration and the Michaelis-Menten kinetics fitting was performed using the following equation:

$$ν = {k}_{cat} {E}_{0} \frac{[ S ]}{{K}_{M} + [ S ]} \qquad ( 5 )$$

where k_{cat} is the catalytic turnover rate of the enzyme, E₀ is the activated enzyme concentration (1 nM in this experiment), [S] is the substrate concentration, and K_{M} is the Michaelis-Menten constant.

RPA/RT-RPA. RPA was performed using the TwistAmp Basic kit. For RT-RPA amplifying HIV RNA, each 50 μL reaction included 29.5 μL of rehydration buffer, 500 nM of forward primer, 500 nM of reverse primer, 4 U/μL of SuperScript IV Reverse Transcriptase, 0.15 U/μL of RNase H, 280 mM of magnesium acetate (provided in the TwistAmp Basic kit), and 5 μL of extracted nucleic acid template. The protocol for RT-RPA was 42° C. for 40 min followed by heat inactivation of the RT enzyme and RNase H at 85° C. for 20 min. For amplification of HPV 16 DNA, the RT enzyme and RNase H were not necessary, and the reaction was performed at 39° C. for 20 min. The RPA amplicons were kept frozen until use.

Implementation of the UNIVERSE assay. T7 transcription and CRISPR-based detection were implemented in one pot as a one-step process in the UNIVERSE assay. Each 25 μL reaction in a PCR tube (Bio-Rad Laboratories) was composed of 1×NEBuffer r3.1, 1×RNAPol Reaction Buffer, 5 μM of ssDNA-FQ (/5′6-FAM/TTATT/3′IABKFQ/), 1 mM of Ribonucleotide Solution Mix, 2 U/μL of RNase Inhibitor (Murine), 300 nM of crRNA, 5 U/μL of T7 RNAP, and 300 nM of Cas12a enzyme. Alt-R L.b. Cas12a (Cpf1) Ultra from IDT is preferred in some embodiments of the disclosure. The RPA amplicons were then transferred into the reaction mixture, which was operated in a PCR workstation (AirClean Systems) to avoid cross-contamination. 1.5 μL of RPA amplicons were introduced for detection of plasmid, and 6 μL of RPA amplicons were introduced for detection of plasma positive control and clinical samples. The reaction was then thoroughly mixed, briefly centrifuged, and incubated at 37° C. in the CFX96 Touch Real-Time PCR Detection System (Bio-Rad Laboratories) for 2-3 h of fluorescence monitoring with a recording interval of 15 s.

DNase removal of the RPA amplicons for a control experiment. To run the control experiment, the steps of the UNIVERSE assay were performed separately using the following procedure: 1) RPA reaction on DNA plasmid at 39° C. for 20 min; 2) T7 transcription on the RPA amplicon at 37° C. for 2 h; 3) DNase I-XT treatment (0.08 U/μL) to the transcription product at 37° C. for 15 min; and 4) introduction of the CRISPR reaction and real-time fluorescence monitoring at 37° C. for 2 h. Two Cas12a orthologs (AsCas12a Ultra and LbCas12a Ultra from IDT) and two template concentrations (3×106 copies/μL and 3×104 copies/μL) were investigated. The control group was not treated with DNase, and the same volume of nuclease-free water was added in Step 3. The concentrations of the CRISPR reaction components were kept the same as those used in the UNIVERSE assay, described above.

Gel electrophoresis. Denaturing polyacrylamide gel electrophoresis (PAGE) (10%) was performed to analyze cis-cleavage on both ssDNA and ssRNA targets with different enzymes and to verify the existence of RPA amplicons and transcription products after applying T7 RNAP. To prepare the gel, 9.6 g urea was dissolved in a mixture of 5 mL of 40% acrylamide solution and 2 mL of 10×TBE buffer. Deionized water was added to bring the volume to 20 mL. Then, 66 μL of 30% APS solution and 8 μL of TEMED solution were added into the gel mixture as the catalysts for gel polymerization. The mixture was then transferred to the gel-casting chamber before it solidified. To verify the RPA amplicons and transcription products, ss20 DNA ladder was loaded in the first lane of the gel as the reference for sizes of denatured nucleic acids. All samples contained 1× Invitrogen Gel Loading Buffer II. The running time was 60 min, with the voltage held constant at 200 V and the initial current set as 15 mA. Following completion of the electrophoresis, the gel was stained in a dark environment for 20 min with SYBR Gold Nucleic Acid Gel Stain which was dissolved to 1×in 1×TBE buffer. For the cis-cleavage assay using FAM-labeled targets, SYBR Gold staining was not necessary. Gel images were obtained with the Bio-Rad ChemiDoc MP Imaging system.

Clinical sample preparation and qPCR/RT-qPCR detection. HIV clinical plasma samples were obtained and inactivated in the clinical microbiology laboratory at the University of Connecticut Health Center. The HIV RNA was extracted from clinical plasma samples as well as the AcroMetrix HIV High Control using the QIAamp Viral RNA Mini Kit. The HPV 16 DNA was extracted from clinical cervical swab samples using the DNeasy Blood and Tissue Kit. The nucleic acid extraction was performed according to the manufacturer's protocol. Real-time qPCR/RT-qPCR was used as a standard for identification of positive and negative clinical samples. The qPCR/RT-qPCR assay was prepared using the GoTaq Probe 1-Step RT-qPCR kit. For RT-qPCR in the detection of HIV RNA, one 20 μL reaction contained 1× GoTaq qPCR Master Mix, 1× GoScript RT Mix for 1-Step RT-qPCR, 500 nM of forward primer, 500 nM of reverse primer, 200 nM of TaqMan probe, and 2 μL of extracted HIV RNA. The protocol of RT-qPCR included the following steps: 1) reverse transcription at 45° C. for 15 min; 2) RT inactivation and hot-start polymerase activation at 95° C. for 10 min; 3) 40 cycles of denaturation at 95° C. for 10 s, annealing at 60° C. for 30 s and extension at 72° C. for 30 s. For qPCR in the detection of HPV 16 DNA, GoScript RT Mix for 1-Step RT-qPCR was eliminated and the other reaction components remained the same. The protocol excluded the reverse transcription step, and the other steps remained the same. Fluorescence was monitored using the CFX96 Touch Real-Time PCR Detection System (Bio-Rad Laboratories) with a recording interval of 15 s.

Digital RT-PCR. Digital RT-PCR was performed using the QuantStudio 3D Digital PCR System. Each RT-PCR reaction was in a total volume of 34.8 μL and contained 1× Reliance One-Step Multiplex Supermix, 900 nM of forward primer, 900 nM of reverse primer, 250 nM of TaqMan probe, and 3.48 μL of RNA template extracted from the positive control. Then, 14.5 μL of the RT-PCR reaction mixture was loaded into the 3D Digital PCR chip, which was included in the QuantStudio 3D Digital PCR 20K Chip Kit v2 (Applied Biosystems). The packaging of the chip was achieved using a 3D Digital PCR Chip Loader (Applied Biosystems) according to the manufacturer's instructions. The chip was then placed on the ProFlex 2× Flat Block Thermal Cycler (Applied Biosystems) and incubated using the following protocol: 1) reverse transcription at 50° C. for 10 min; 2) RT inactivation and DNA polymerase activation at 96° C. for 10 min; 3) 40 cycles of annealing/extension at 56° C. for 2 min and denaturation at 98° C. for 30 s; 4) final extension at 56° C. for 2 min; and 5) storage at 10° C. until the chip was read. Once the RT-PCR reaction was complete, the chip was read on either a QuantStudio 3D Digital PCR Instrument (Applied Biosystems) or a fluorescence microscope (Axio Observer, ZEISS).

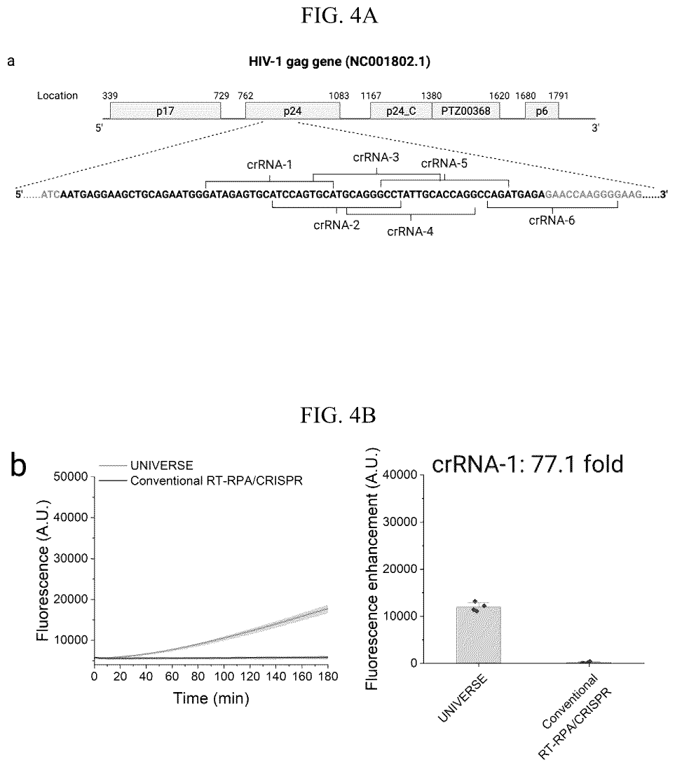

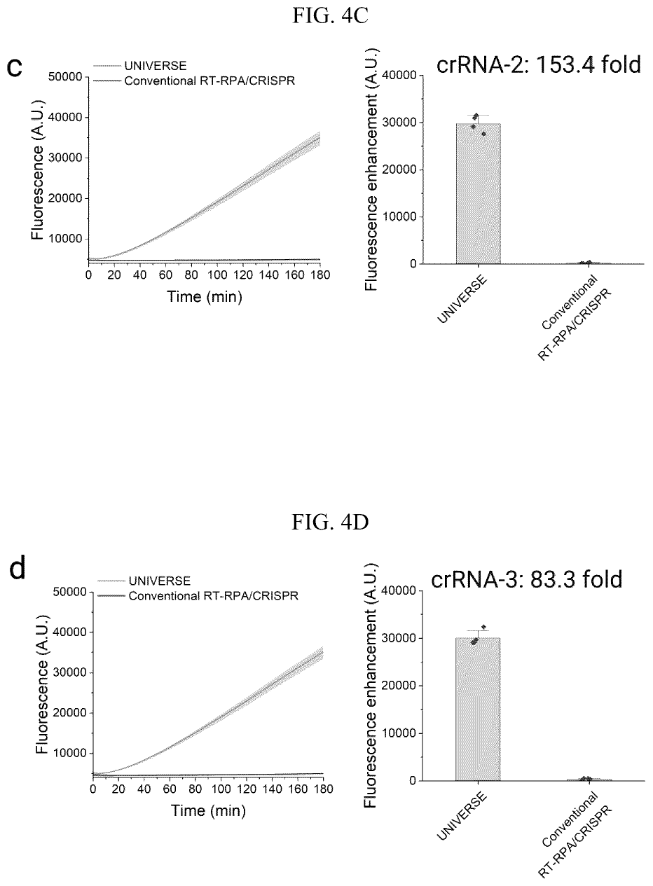

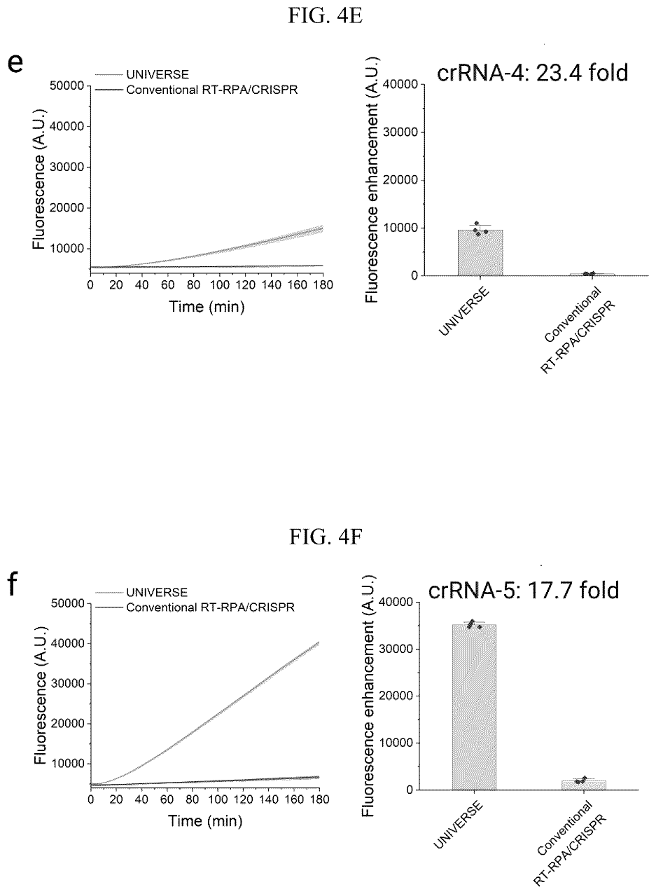

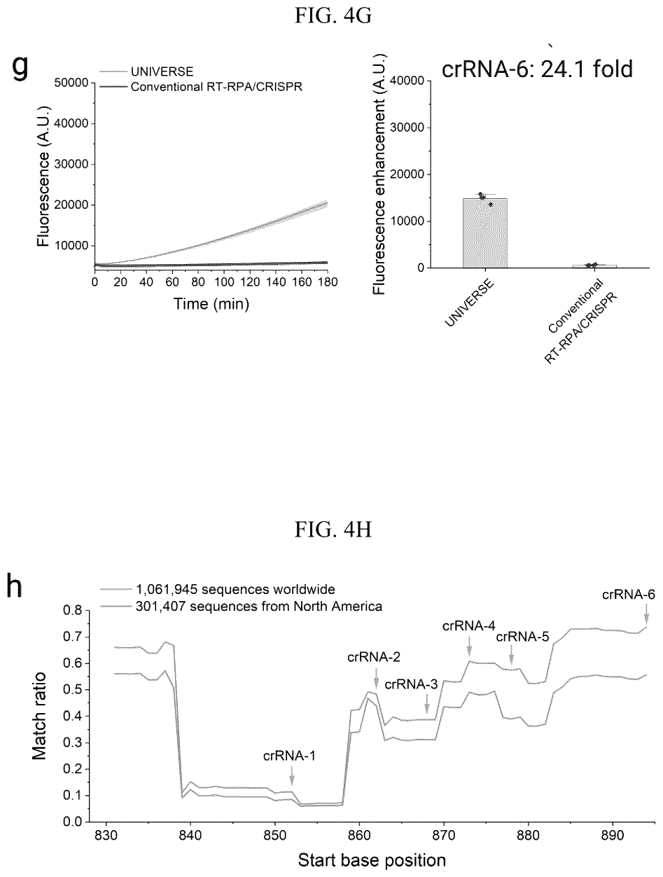

Code availability. The source code implementing the calculation of match ratio for sequence homology screening of the CRISPR target in FIG. 4H was written in Python 3 and is available on GitHub. Examples used in this work are provided along with the code to enable testing of reproducibility. The HIV sequencing records were obtained from the NCBI Virus Database.

### Example 2. RNA-Initiated Trans-Cleavage with CRISPR-Cas12a

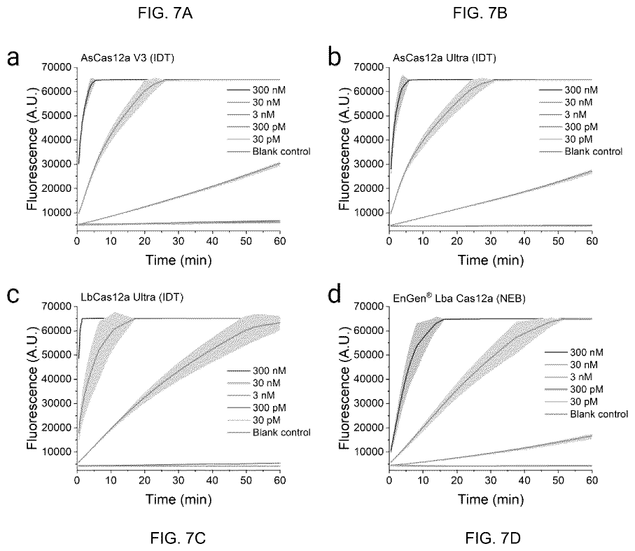

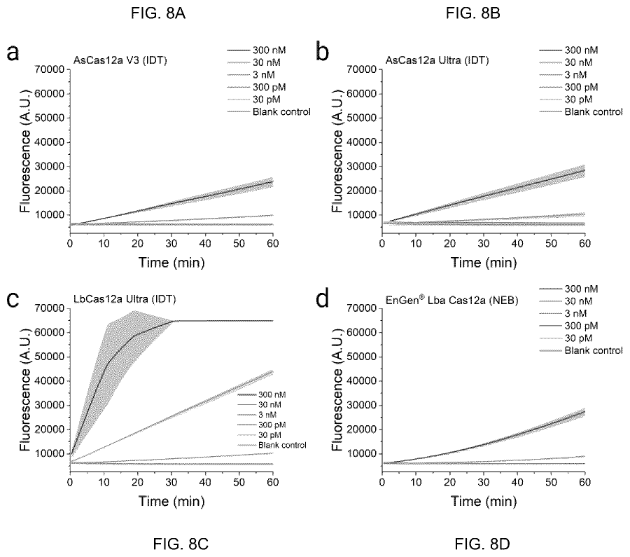

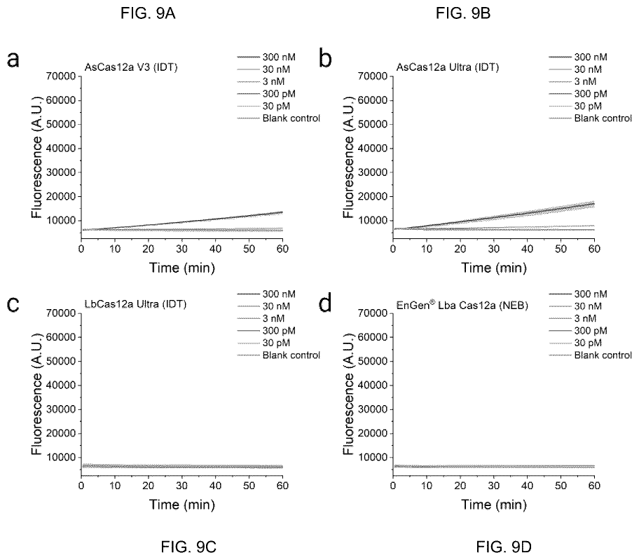

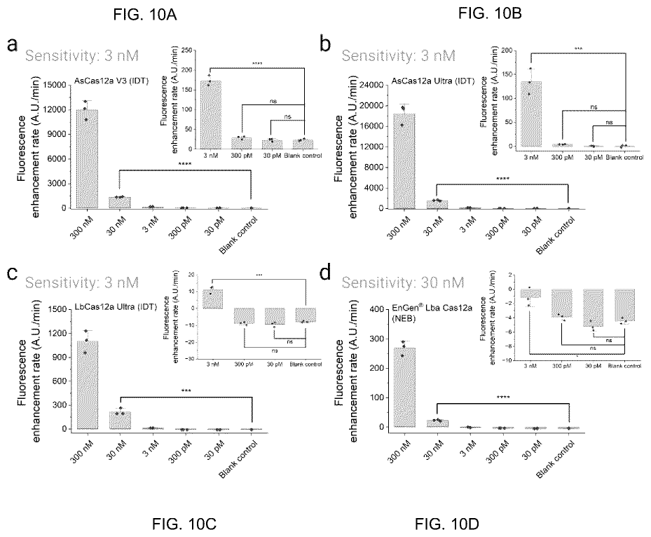

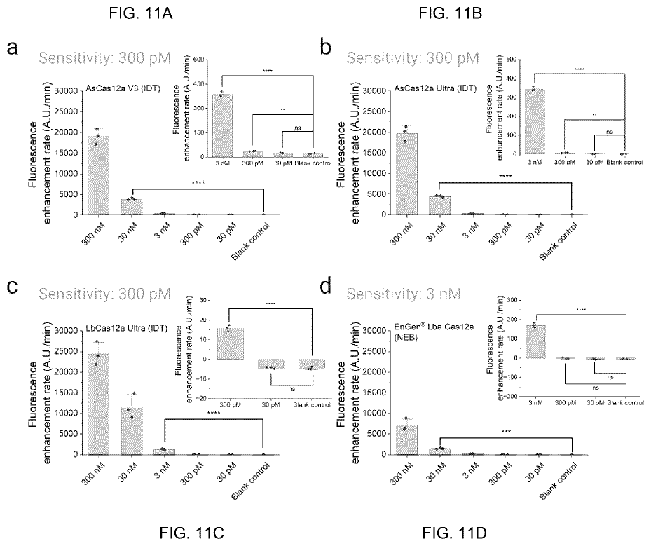

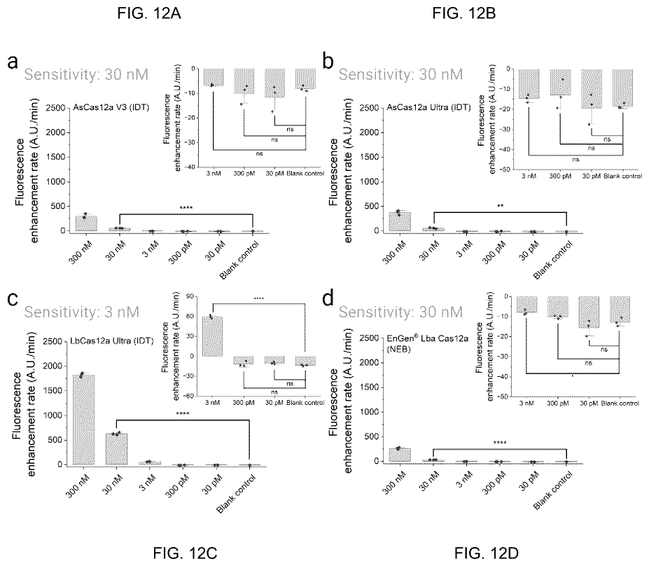

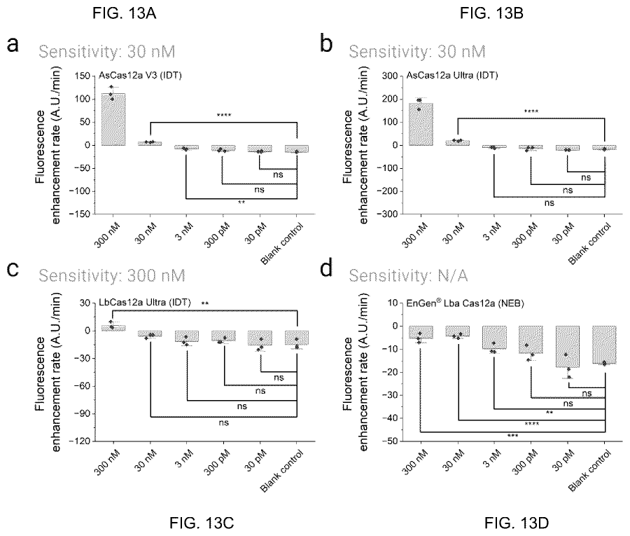

To investigate the intrinsic RNA-targeting characteristics of Cas12a, a fully complementary, 20-nt single-stranded RNA (ssRNA) was designed as Cas12a's target. Experiments were conducted to assess the RNA-activated trans-cleavage activity of four commercially available Cas12a nucleases, including AsCas12a V3, AsCas12a Ultra, LbCas12a Ultra, and EnGen Lba Cas12a. Surprisingly, a significant increase in fluorescence intensity was observed with all four Cas12a nucleases, indicating effective trans-cleavage induced by the RNA targets (See FIG. 1A-1D). In particular, AsCas12a (AsCas12a V3 and AsCas12a Ultra) exhibited significantly higher RNA-activated trans-cleavage activity compared to LbCas12a (LbCas12a Ultra and EnGen Lba Cas12a). Additional experiments were conducted to determine the Michaelis-Menten kinetics of AsCas12a. Catalytic efficiencies (k_{cat}/K_{M}) were obtained of 2.1×10⁴ M⁻¹ s⁻¹ for AsCas12a V3 and 5.4×10⁴ M⁻¹ s⁻¹ for AsCas12a Ultra when activated by ssRNA (FIG. 6). In order to compare the trans-cleavage efficiency of the ssRNA target with traditional DNA target, a 20-nt ssDNA target was designed with an identical sequence to the ssRNA and a trans-cleavage assay was performed (FIG. 7). Additionally, the fluorescence intensity of trans-cleavage on the RNA substrate was measured by switching the ssDNA reporters to ssRNA reporters in the assay (FIG. 8 and FIG. 9). When using RNA targets and ssDNA reporters, a weakening of trans-cleavage activity was observed for the LbCas12a nucleases and a relatively slighter weakening for the AsCas12a nucleases (FIG. 1E). However, significant differences in sensitivity were not observed when detecting both DNA and RNA targets (FIG. 10 and FIG. 11). With ssRNA reporters being the substrates, the trans-cleavage activity of Cas12a sharply declined regardless of RNA or DNA targets, and their sensitivity became correspondingly lower (FIG. 12 and FIG. 13). Thus, the present disclosure provides, in some embodiments, that a full-size ssRNA is useful to trigger trans-cleavage activity of both AsCas12a and LbCas12a nucleases. In some embodiments, trans-cleavage activity is of a lower magnitude for LbCas12a. Thus, the disclosure provides the use of Cas12a-based RNA detection (e.g., by coupling with ssDNA reporter).

Next, it was determined whether cis-cleavage occurs on a ssRNA target. Strong cis-cleavage activity might cut the ssRNA target thereby reducing detection sensitivity. A previous study suggested that the cis-cleavage site for ssDNA was near the 22^{nd} base counting from the 3′ end of the first base pairing with the crRNA (See, S.-Y. Li, et al., Cell Research 2018, 28, 491). Given that the target used in the above example only contained 20 nt, the cis-cleavage site might lie outside the targeting region and within the 5′ extension of the target. Considering potential differences in mechanisms between ssDNA-activated and ssRNA-activated cis-cleavage, two 5′ FAM-labeled ssRNA targets were designed. One was extended at the 5′ end by a 20-nt sequence and the other was extended by 20 nt at both the 5′ and 3′ ends. To avoid any potential interference from non-specific sequence segments, the extension sequences were designed to ensure there was only one valid targeting site for Cas12a throughout the complete sequence. For the cis-cleavage analysis, wild-type AsCas12a V3 was used, as its mutant AsCas12a Ultra exhibited minimal differences in the trans-cleavage assay with ssRNA targets.

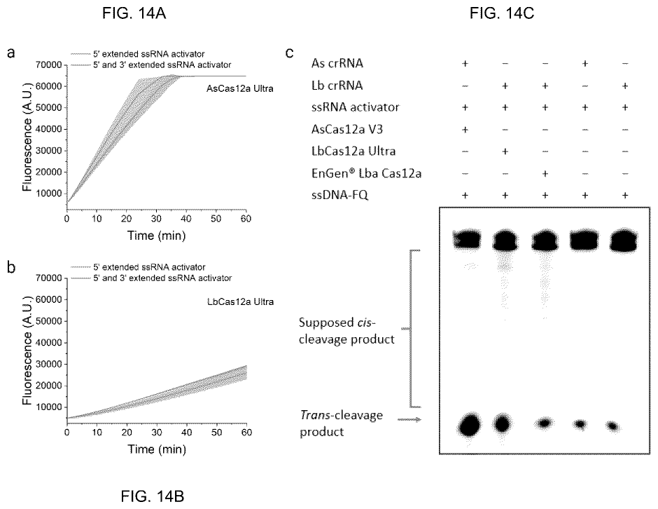

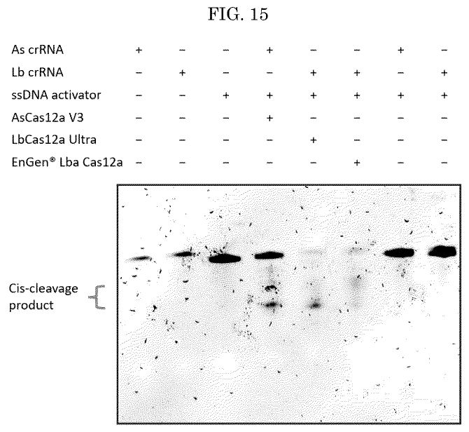

After a 60-min incubation at 37° C., neither AsCas12a nor LbCas12a exhibited cis-cleavage on 5′-extended ssRNA targets or on 5′- and 3′-extended ssRNA targets (FIG. 1F), indicating that Cas12a could not cleave the ssRNA targets. To further characterize this finding, a 3′ FAM-labeled ssRNA target was designed with both 5′ and 3′ extensions and the cis-cleavage assay performed, obtaining results consistent with the original finding (FIG. 14E). In contrast, the cis-cleavage was apparent on ssDNA targets with only a 5′ extension for all of the enzymes tested (FIG. 15). LbCas12a nucleases almost completely cleaved ssDNA targets within a 60-min incubation period, whereas AsCas12a nucleases cleaved ssDNA targets to a lesser extent, albeit noticeably stronger than the cis-cleavage activity observed with ssRNA targets. Thus, the present disclosure provides that both cis-cleavage and trans-cleavage activities on ssRNA substrates were negligible with LbCas12a and AsCas12a.

Thus, the disclosure identified and characterized the properties of three type V single-effector Cas nucleases including two Cas12a orthologs (i.e., LbCas12a and AsCas12a) commonly used for DNA-activated nucleic acid detection and one newly discovered RNA-activating nuclease, SuCas12a2. The disclosure identifies that their effective nucleic acid types as activators, cis-cleavage substrates, and trans-cleavage substrates (See FIG. 1G). For LbCas12a, both ssDNA and dsDNA can initiate nuclease activity, leading to degradation of the DNA activators themselves and collateral ssDNA or dsDNA substrates. AsCas12a possesses the same nuclease activity as LbCas12a, with the exception of the trans-cleavage on dsDNA substrates. Unique from LbCas12a and AsCas12a, SuCas12a2 required a protospacer flanking site (PFS) on its RNA activator (5′-GAAAG-3′) to trigger trans-cleavage activity, and this collateral degradation applies to ssDNA, dsDNA, and ssRNA. Because SuCas12a2 lacks the Nuc domain involved in DNA target strand loading, DNA activators may not be capable of initiating the nuclease activity. In addition, the degradation of RNA target with Cas12a2 was mild enough to exhibit a distinct difference from the cis-cleavage of DNA targets with Cas12a. Thus, the disclosure provides that ssRNA target can initiate trans-cleavage activity of both AsCas12a and LbCas12a, with LbCas12a displaying relatively lower trans-cleavage activity. ssRNase activity was consistently weaker than ssDNase activity for both LbCas12a and AsCas12a.

### Example 3. RNA Activation Improves the Detection Specificity of Cas12a

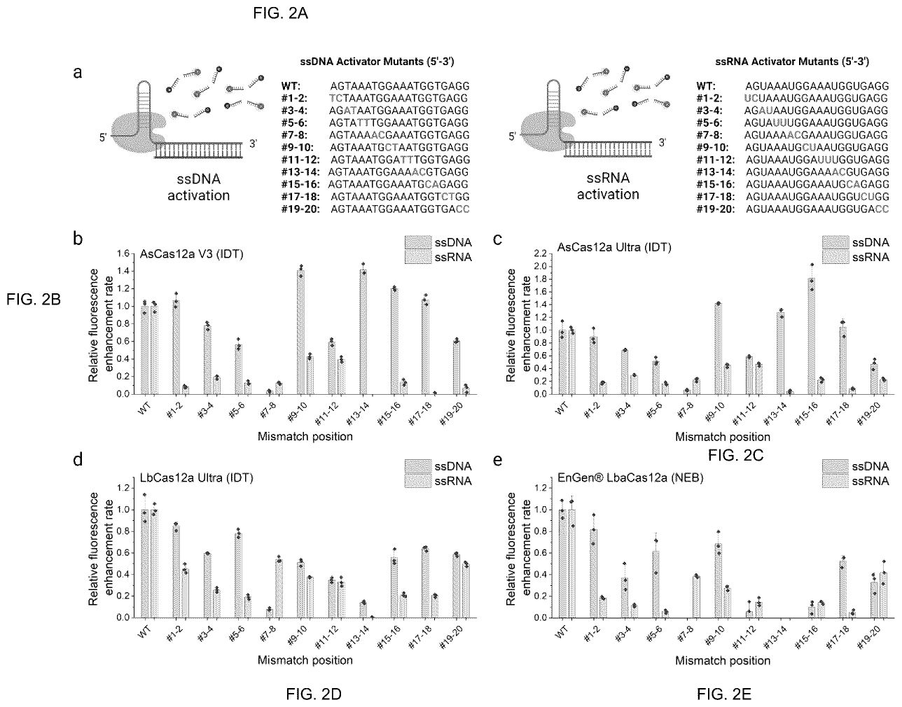

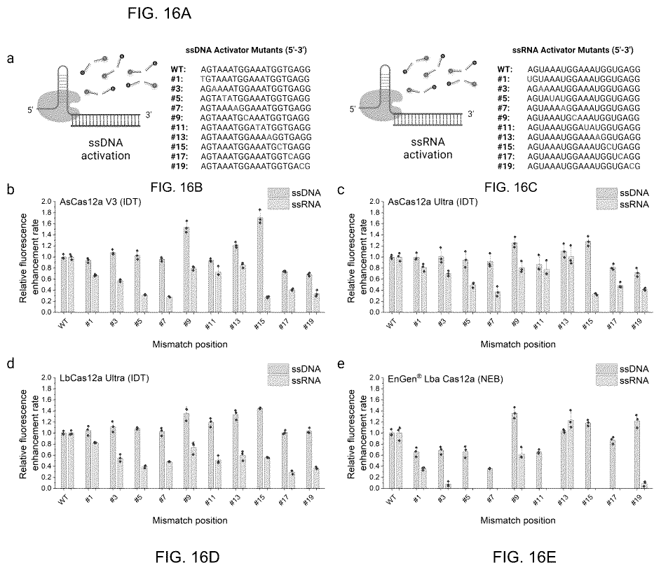

In order to further characterize the discovery of the intrinsic RNA-targeting ability of Cas12a, it was determined whether there might be a compromise in target recognition specificity when switching the target from DNA to RNA. Experiments were conducted that utilized the design of a series of 20-nt targets containing continuous two-base mismatches, where the mismatches were the complementary bases to the fully matched ones (See FIG. 2A). The specificity of target recognition was determined by calculating the relative fluorescence enhancement rate between a mismatched target and a fully matched target. A lower relative fluorescence enhancement rate indicates better specificity because the mismatched target is more likely to be distinguished. The concentration of ssDNA or ssRNA targets was maintained at 30 nM for all enzymes tested. As seen in FIGS. 2B-2E, mismatched ssRNA targets generally had a weaker fluorescence response compared to mismatched ssDNA targets for all four Cas12a enzymes tested. Specifically, only one group of mismatched ssRNA targets (mismatches #7-8) exhibited similar or higher relative fluorescence enhancement rates compared to their ssDNA counterparts (See FIGS. 2B, 2C, and 2D). Additionally, for the ssDNA targets, there were several cases in which the fluorescence response for a mismatched target was even stronger than that for the fully matched one, indicating a failure to identify a mismatched ssDNA target. However, the fluorescence responses for the fully matched ssRNA target were consistently higher than those for the mismatched ssRNA targets for all enzymes tested, indicating that the mutated RNA targets could always be distinguished. Specificity tests on single-base mismatches yielded the same conclusions (See FIG. 16). Therefore, the disclosure provides that RNA activation improves the specificity of trans-cleavage for both Cas12a orthologs tested, rendering it a more ideal option for nucleic acid detection, especially when a higher level of specificity is required.

### Example 4. Highly Sensitive PAM-Free Nucleic Acid Detection Using UNIVERSE

Based on the discovery, described above, of the trans-cleavage activity of Cas12a triggered by RNA targets, additional experiments were conducted in an effort to develop a universal nucleic acid detection method by combining T7 transcription of DNA amplicons with this newly discovered RNA-activated trans-cleavage feature of Cas12a. The nuclease activity of Cas12a revealed that a protospacer adjacent motif (PAM), while required for strand separation in double-stranded targets, was not necessary for successful recognition of single-stranded targets. Considering that transcripts are released from the enzyme at the termination site of the DNA template, the product of T7 transcription was expected to be ssRNA. As disclosed herein, ssRNA can be recognized without PAM restriction thereby allowing the generation of a PAM-free CRISPR-based detection system.

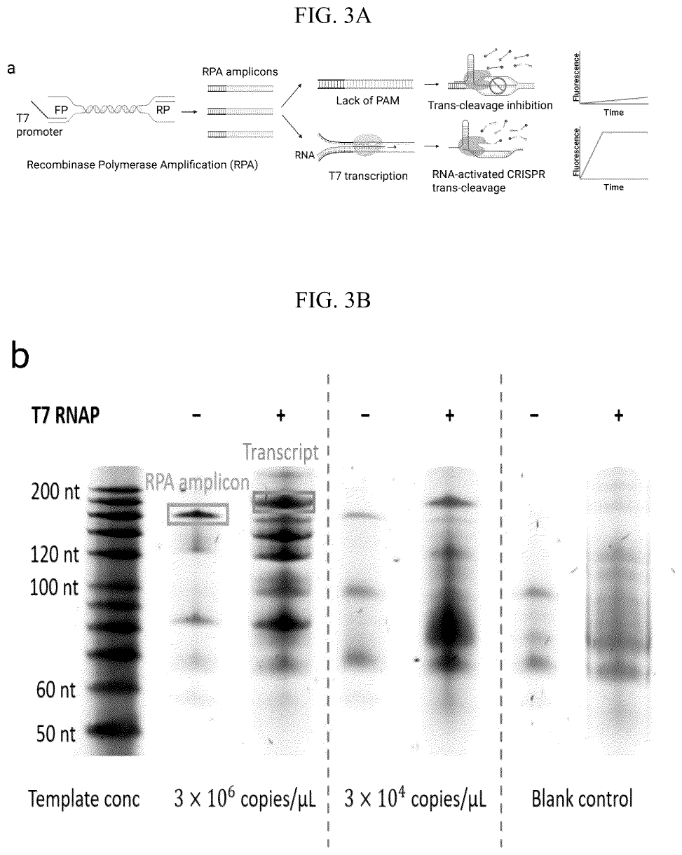

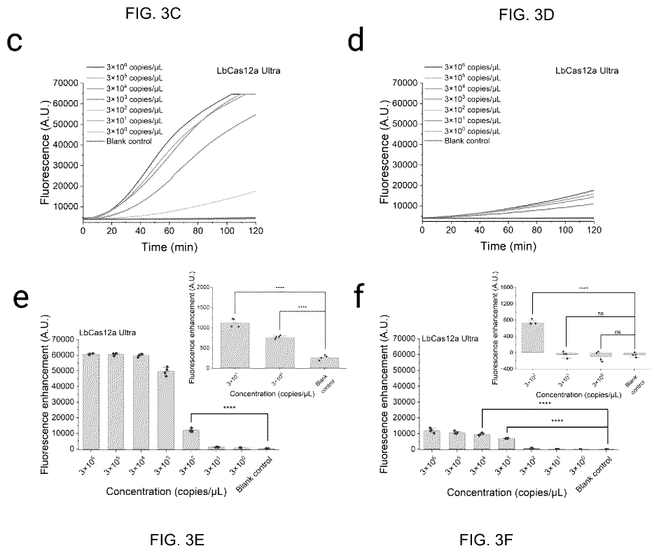

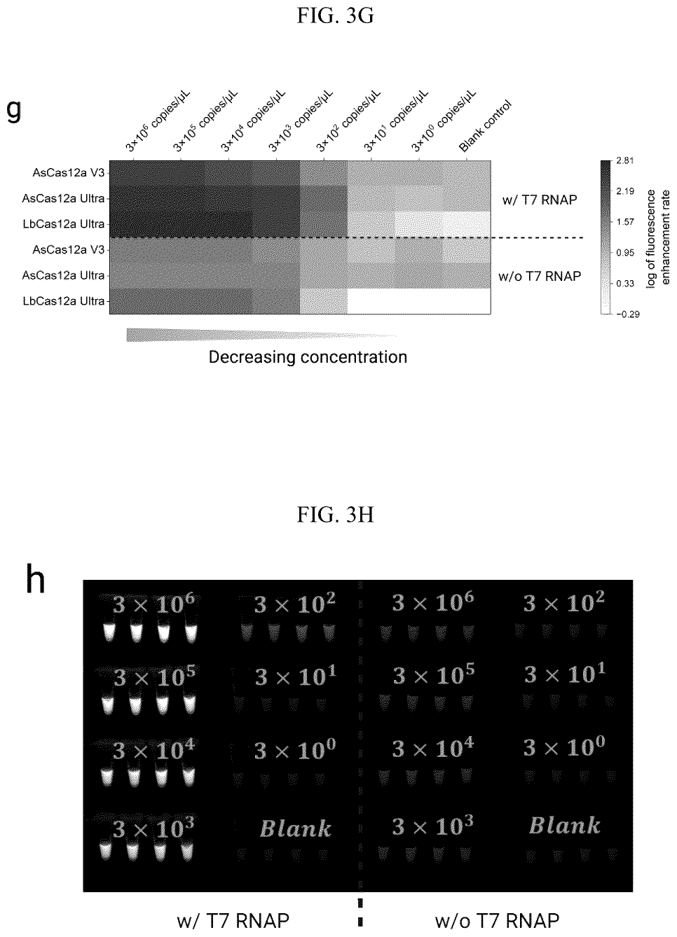

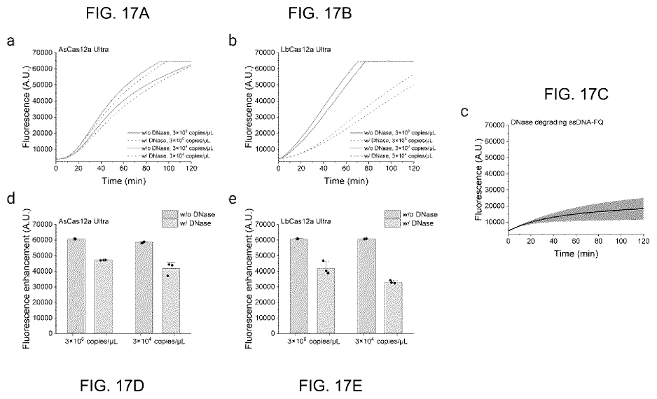

T7 transcription unwinds the dsDNA amplicon and produces displaced ssDNA, which also contains the target sequence and contributes to the trans-cleavage signal (FIG. 17). A nucleic acid template was first amplified through an RPA reaction, in which the forward primer was tagged with a T7 promoter, and the amplicons were subjected to T7 transcription for production of RNA targets (FIG. 3). Because the 20-nt target in the RPA amplicon lacked the PAM sequence, LbCas12a and AsCas12a did not efficiently degrade the dsDNA amplicons as the template for RNA synthesis thereby enabling T7 transcription and CRISPR-based detection to proceed in one pot (e.g., in one reaction vessel or tube). This reaction scheme was coined “Universal Nuclease for Identification of Virus Empowered by RNA-Sensing” (UNIVERSE, also referred to herein as the “UNIVERSE ASSAY” or “UNIVERSE SYSTEM”). Without T7 transcription, the PAM-less dsDNA target does not induce significant fluorescence enhancement from the trans-cleavage activity of the Cas12a orthologs. In contrast, the fluorescence response dramatically improves with the incorporation of a T7 transcription step (FIG. 3A). Denaturing gel electrophoresis was performed to verify the production of transcripts by T7 transcription. RPA amplicons, in the presence of T7 transcription, were almost completely transcribed to RNA by T7 RNA Polymerase (T7 RNAP, FIG. 3), although this process generated numerous by-products, which could potentially be transcripts from non-specific RPA amplicons (FIG. 3B).

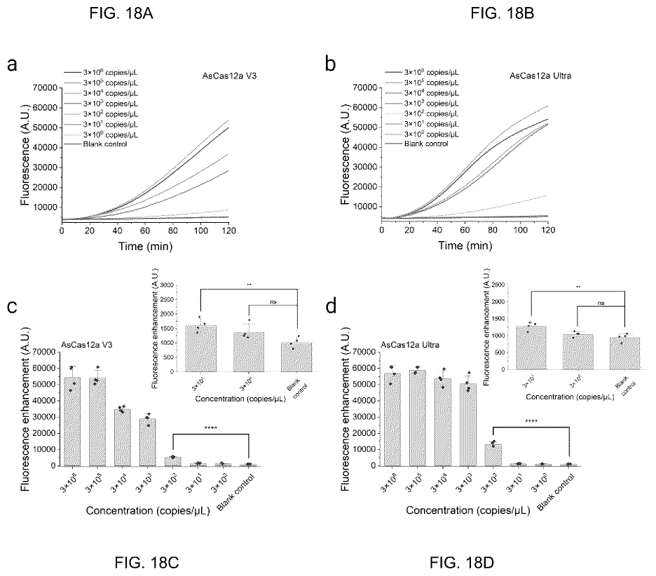

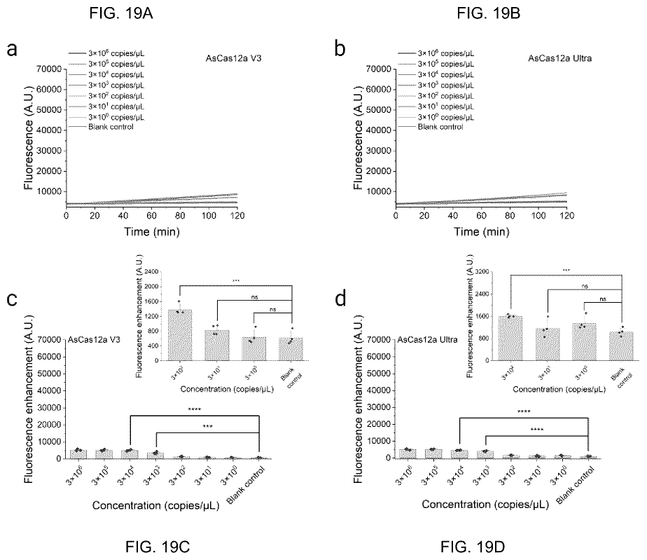

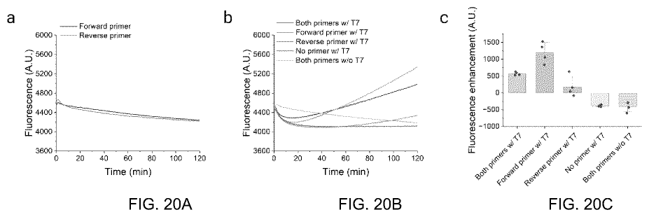

Next, experiments were conducted in an effort to adapt UNIVERSE to carry out a trans-cleavage assay by monitoring the real-time fluorescence generated from degradation of ssDNA-FQ. The performance of the UNIVERSE assay was examined and characterized using a plasmid containing the p24 gene of the HIV genome. The CRISPR target was a 20-nt segment within the RPA amplicon, where no PAM sequence was found upstream or downstream. Because EnGen Lba Cas12a exhibited relatively weaker trans-cleavage activity when activated by ssRNA, it was not considered a candidate enzyme to develop the UNIVERSE assay. For the other three enzymes tested, all assays showed near-saturated fluorescence signal within 120 min for a template concentration greater than 3×10³ copies/μL (FIG. 3E and FIG. 18). In sharp contrast, for the conventional RPA/CRISPR-Cas12a assay detecting the same PAM-less target without T7 RNAP, the fluorescence signals remained weak even when high template concentrations were used (FIG. 3D and FIG. 19), likely attributable to the lack of PAM in the CRISPR target sequence. In the UNIVERSE assay, background signals were observed from blank controls with all enzymes tested (See FIG. 3E and FIGS. 18C-18D), which may be a result of non-specific amplification of RPA (FIG. 20). Among the three Cas12a enzymes tested, LbCas12a Ultra showed intermediate RNA-initiating trans-cleavage activity in the conventional CRISPR assay, but exhibited the strongest signal in the UNIVERSE assay compared with the other two AsCas12a enzymes. While an understanding of a mechanism is not needed to practice features of the present disclosure and while the present disclosure is not limited to any particular mechanism, in some embodiments, the strength of the RNA-initiating trans-cleavage activity is attributed to the contribution to signal by displaced ssDNA.

Experiments were also performed in order to evaluate and compare the PAM-free detection sensitivity of the UNIVERSE assay to the detection sensitivity of the conventional RPA/CRISPR-Cas12a assay. For all three enzymes tested, a statistically significant fluorescence enhancement between plasmid samples and blank control was observed at a template concentration of 30 copies/μL with AsCas12a V3 and AsCas12a Ultra (FIG. 18). The UNIVERSE assay achieved a sensitivity as low as 3 copies/μL with LbCas12a Ultra (FIG. 3E). In sharp contrast, for the conventional RPA/CRISPR-Cas12a assay, the highest sensitivity achieved was 300 copies/μL for all enzymes tested (FIG. 3F and FIG. 19). Thus, in some embodiments, the disclosure provides that the UNIVERSE assay provides about a 100-fold improvement in detection sensitivity compared to conventional RPA/CRISPR-Cas12a assays.

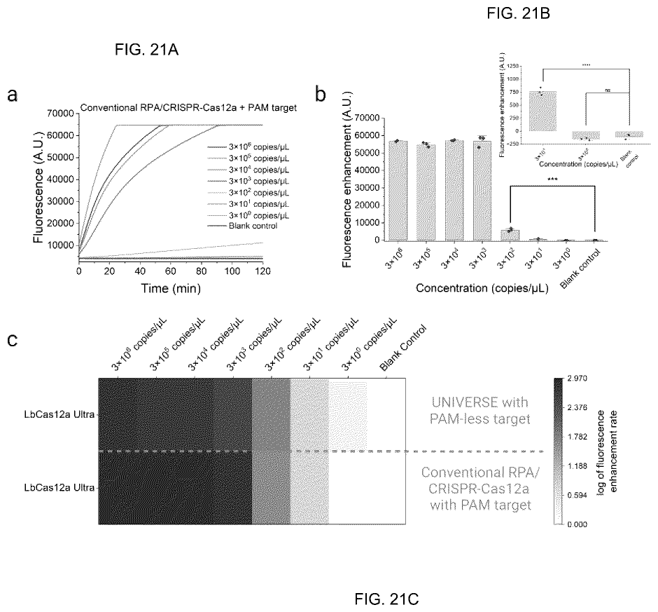

In order to further characterize these findings, the base 10 logarithm of the average fluorescence enhancement rates within the first 60 min of the reaction were calculated (FIG. 3G). The addition of T7 RNAP significantly accelerated the fluorescence enhancement in the RPA/CRISPR-Cas12a assay. More importantly, the UNIVERSE assay generated much stronger endpoint fluorescence signals compared to the conventional RPA/CRISPR-Cas12a assay, enabling direct visual detection that was not achieved with the conventional RPA/CRISPR-Cas12a assay (See FIG. 3H). While detection of a PAM target was efficient with the conventional RPA/CRISPR-Cas12a assay when the template concentration was high, at low template concentrations that are typically encountered in real-world pathogen (e.g., viral) testing, the UNIVERSE assay disclosed herein (e.g., that detects a PAM-less target) exhibited an efficiency similar to that of a conventional RPA/CRISPR-Cas12a assay with high template concentration (FIG. 21). Thus, in some embodiments, the disclosure provides the UNIVERSE assay that has a significantly higher sensitivity than conventional RPA/CRISPR-Cas12a assays (e.g., the UNIVERSE assay displays significantly better nucleic acid detection in terms of magnified signal intensity and improved detection sensitivity compared to conventional RPA/CRISPR-Cas12a assay).

### Example 5. UNIVERSE Enables Unrestricted Selection of Target Sequences

Prior to the present disclosure, Cas12a-based nucleic acid detection has been inherently limited by the requirement of PAM or suboptimal PAM sequences. However, assays discovered during embodiments of the present disclosure and described herein (e.g., the UNIVERSE assay) indicated that any nucleic acid sequence (e.g., a nucleic acid sequence lacking a PAM or suboptimal PAM sequence) could be used to initiate the trans-cleavage response of Cas12a (e.g., ssRNA and/or displaced ssDNA without a PAM sequence could be used to activate the trans-cleavage response of Cas12a). While an understanding of a mechanism is not needed to practice embodiments of the disclosure and while the disclosure is not limited to any particular mechanism of action, it was tested whether the UNIVERSE assay could eliminate the requirement for PAM or suboptimal PAM sequences to attain unrestricted selection of target sequence(s) in an amplicon. To test this hypothesis, six 20-nt CRISPR target sequences were arbitrarily chosen from the amplicon of the HIV detection mentioned above. These targets were screened to exclude canonical and suboptimal PAM sequences. The positions of the targets are labeled shown in FIG. 4A. Using nucleic acids extracted from the HIV RNA-positive plasma control, both conventional RT-RPA/CRISPR-Cas12a and the UNIVERSE assay were performed. In the conventional RT-RPA/CRISPR-Cas12a assay, all six targets generated negligible or very weak fluorescence signal over a 3 h incubation. However, the fluorescence signal was remarkably enhanced in the UNIVERSE assay, with a 17.7-fold to 153.4-fold change in fluorescence enhancement compared to the conventional RT-RPA/CRISPR-Cas12a assay (FIGS. 4B-4G).

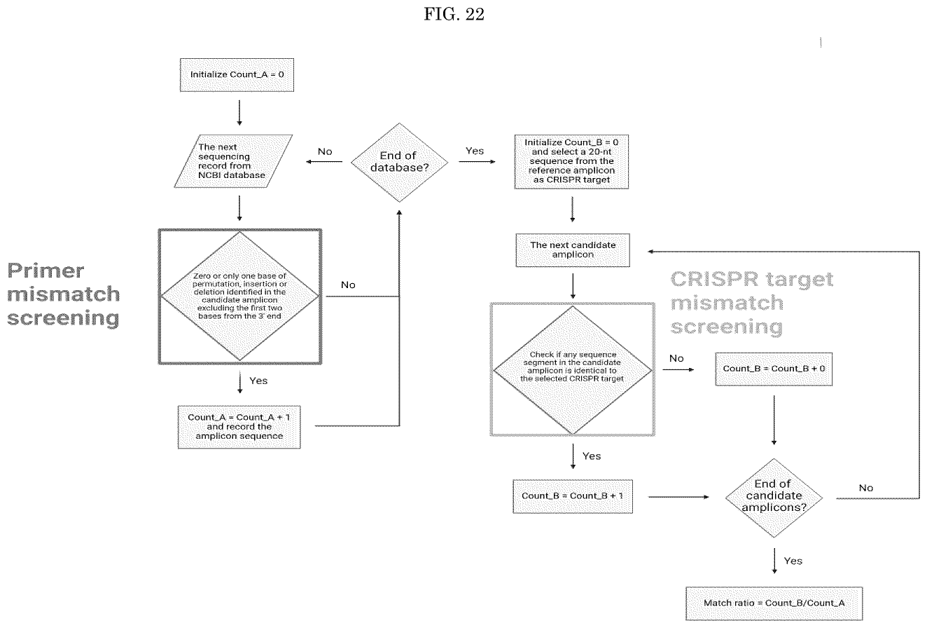

Next, it was determined whether these target sequences are more homologous by comparing the selected sequence to 1,061,945 HIV sequencing records worldwide from the NCBI Virus Database (FIG. 22, See E. L. Hatcher, et al., Nucleic Acids Research 2017, 45, D482). Considering that the RPA reaction remains effective in the presence of a single-base mutation, insertion, or deletion not located close to the 3′ ends of the primers, 43,320 valid amplicons were obtained from the sequencing records using the described primer pair. Within these amplicon sequences, every 20-nt segment within the amplicon sequences were searched and the records counted with a perfect match to the sequence of the selected CRISPR target. The homology was represented by a match ratio, indicating the proportion of matching records to the total number of valid amplicons. In view of the fact that the HIV clinical samples were collected in North America, and considering the possibility of geographical specificity for viral mutations, a screening of records in North America was also conducted with 301,407 samples. Most of the selected targets were observed to be in regions with higher homology (FIG. 4H), which established the basis of using the corresponding crRNAs for clinical detection. Next, detection on clinical HIV-positive samples was performed, validating the clinical effectiveness of the UNIVERSE assay through unrestricted selection of CRISPR targets. Similar to the detection of the HIV-positive control, there was a significant improvement in fluorescence response compared to the conventional RT-RPA/CRISPR-Cas12a assay (FIG. 4I). Thus, in some embodiments, the disclosure provides that the UNIVERSE assay not only broadens the range for target sequence selection, but also increases the fluorescence detection signals. Accordingly, the present disclosure provides that the UNIVERSE assay displays a higher sensitivity, is more robust, and more reliably detects nucleic acid than conventional RT-RPA/CRISPR-Cas12a assays.

### Example 6. Viral Nucleic Acid Detection in Clinical Samples Using UNIVERSE

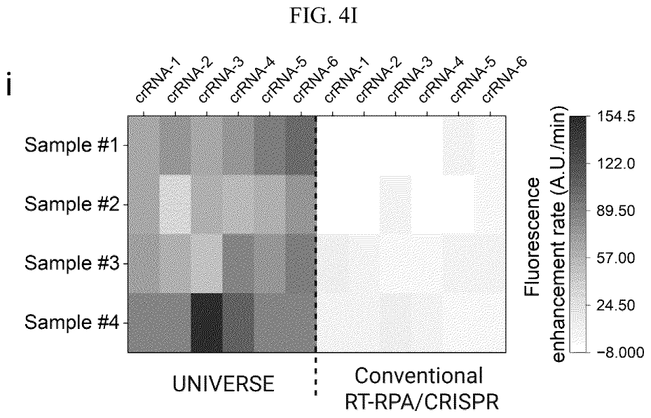

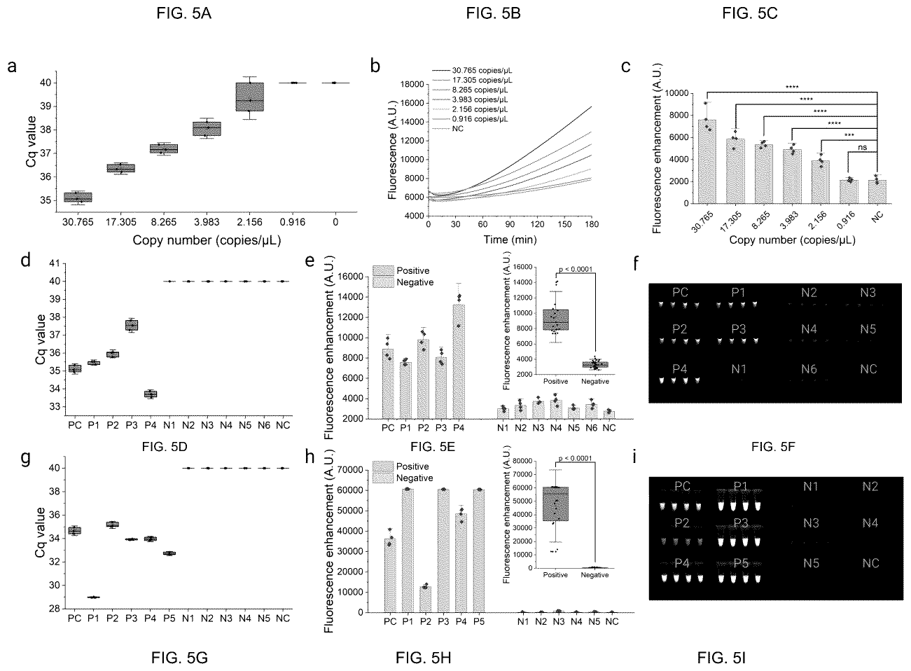

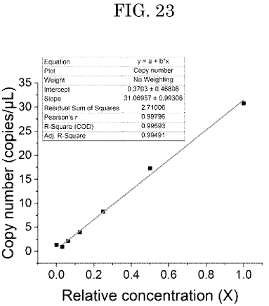

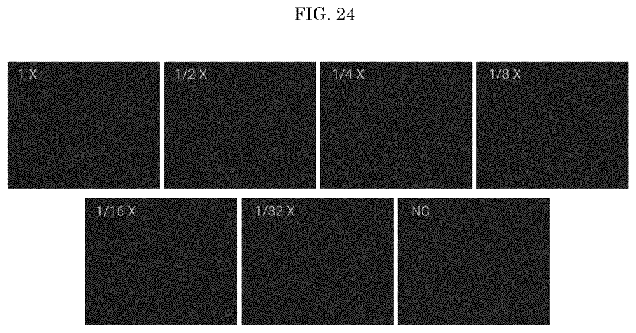

Before testing HIV clinical samples, detection sensitivity was assessed using an HIV-positive control in human plasma. To enable accurate sensitivity evaluation, HIV RNA copy numbers in the HIV-positive control samples were quantified using digital RT-PCR (FIG. 23 and FIG. 24). Additionally, real-time RT-qPCR, the gold standard for HIV detection, was used to detect HIV in serially diluted HIV-positive control samples (FIG. 5A). Then, UNIVERSE assay was used to detect HIV RNA in the serially diluted HIV-positive samples, achieving the same sensitivity (2 copies/μL≈3.3 aM) as the RT-qPCR assay (FIGS. 5B-5C). As shown in Table 1, below, UNIVERSE achieved sensitivity at the attomolar level (e.g., in a PAM-free environment using Cas12a), similar to sensitivities reported for DETECTR (See J. S. Chen, et al., Science 2018, 360, 436) and SHERLOCK (See M. J. Kellner, J et al., Nature Protocols 2019, 14, 2986) assays.

TABLE 1
Comparison of nucleic acid detection assays using CRISPR-Cas systems.

| | | | PAM- | |
|---|---|---|---|---|
| Assay | Nuclease | Mechanism | limitation | Sensitivity |
| DETECTR^{[2]} | LbCas12a | Targeting amplicons in dsDNA | Yes | 1 aM |
| | (Lachnospiraceae bacterium | form, cleaving ssDNA-FQ | | |
| | ND2006 Cas12a) | probes | | |
| SHERLOCK^{[3]} | LwaCas13a (Leptotrichia | Targeting T7-transcribed | No | 2 aM |
| | wadei Cas13a) | amplicons in ssRNA form, | | |
| | AsCas12a (Acidaminococcus | cleaving ssRNA-FQ probes | | |
| | sp. BV3L6 Cas12a) | | | |
| UNIVERSE | LbCas12a Ultra from IDT | Targeting T7-transcribed | No | 2 copies/ |
| | (Recombinant engineered | amplicons in ssRNA form while | | μL ≈ 3.3 aM |
| | Lachnospiraceae bacterium | targeting displaced ssDNA | | |
| | ND2006 Cas12a | during T7-transcription, | | |
| | | cleaving ssDNA-FQ probes | | |

Next, experiments were conducted in order to determine the potential utility of the UNIVERSE assay to detect nucleic acids extracted from clinical samples. Ten clinical plasma samples were tested including four HIV-positive and six HIV-negative samples confirmed by standard RT-qPCR (FIG. 5D). Using the UNIVERSE assay, it was possible to detect and differentiate all HIV-positive samples from negative samples, with confirmation attained using RT-qPCR (FIG. 5E). All positive samples showed significantly higher fluorescence enhancement compared to that of negative samples. The endpoint fluorescence results of clinical HIV detection were visualized using a fluorescence imager, permitting clear differentiation between positive and negative results (FIG. 5F).

Experiments were also conducted to characterize whether the UNIVERSE assay could be used to detect DNA viruses (e.g., HPV 16) from clinical cervical swab samples. Ten clinical cervical swab samples were tested, including five HPV 16-positive samples and five HPV 16-negative samples determined by RT-qPCR (FIG. 5G). After 2 h of incubation, the UNIVERSE assay displayed consistent and clearly distinguishable positive and negative results (FIG. 5H), that could be visually recognized under a fluorescence imager (FIG. 5i).

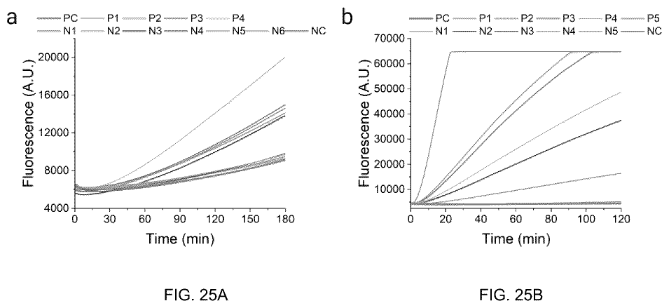

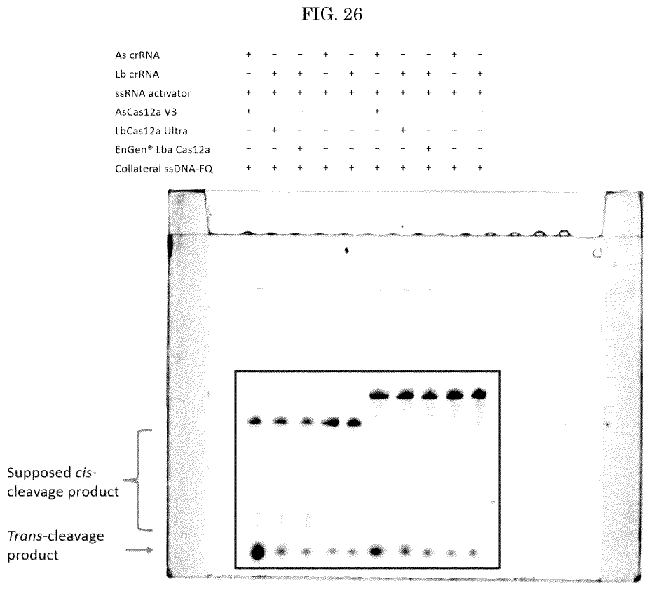

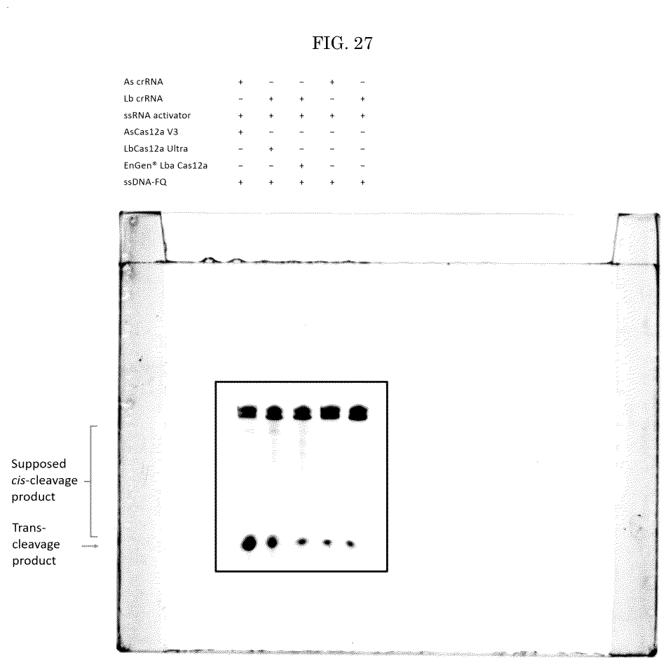

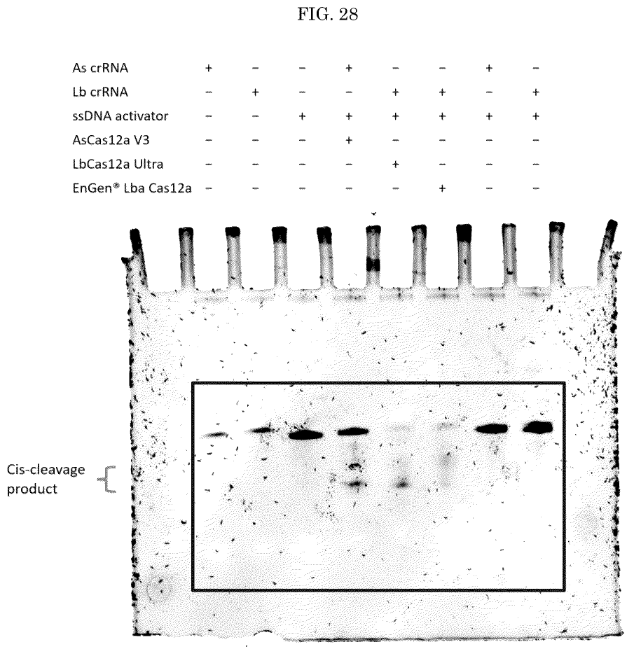

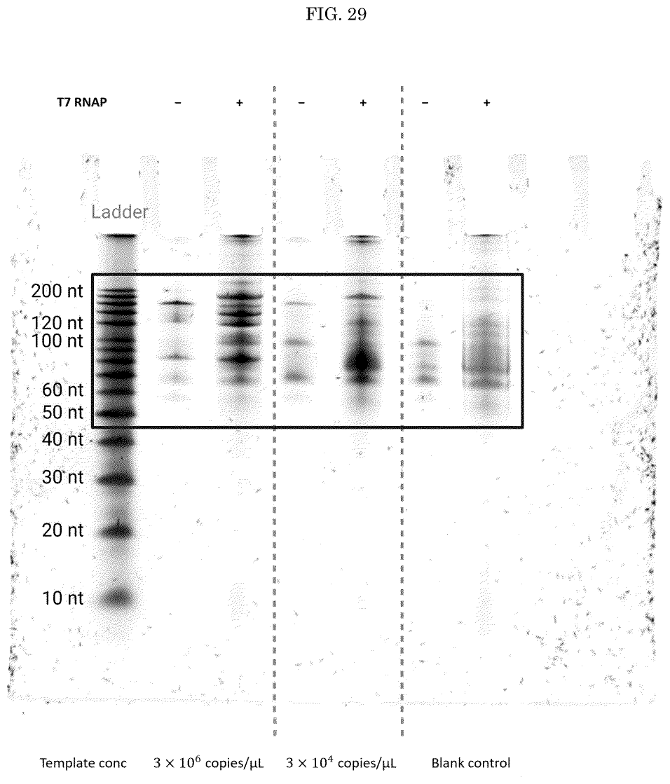

From the real-time fluorescence kinetics, a clear differentiation between positive and negative results was observed at around 75 min for the detection of clinical HIV samples, and as early as 30 min for the detection of clinical HPV 16 samples (FIG. 25). Although the difference in Cq values were trivial, the positive fluorescence response with the DNA virus was stronger than that observed with the RNA virus. Without being limited to any particular mechanism of action, it is speculated that this phenomenon may be related to a variety of factors within the reaction, such as compromised reactivity of T7 polymerase or Cas12a enzyme resulting from less compatible buffering conditions introduced by solvents in reverse transcriptase or RNase H, inconsistent efficiency of RPA amplification, or differences in trans-cleavage activity associated with the sequences of crRNAs and their targeting sites. Thus, in some embodiments, the disclosure provides a simple, universal, PAM-free CRISPR-based method (e.g., the UNIVERSE assay) for nucleic acid detection (e.g., detection of DNA and/or RNA (e.g., from bacteria, viruses, cancer, etc.).

TABLE 2
Sequences of ssDNA and ssRNA targets for the trans-cleavage assay.
The underlined sequences correspond to the spacer of crRNA and its
targeting region.

| Name | Sequence (5′-3′) | SEQ ID NO. |
|---|---|---|
| crRNA | UAAUUUCUACUCUUGUAGAUCCUCACCAUUUCCAUUUACU | SEQ ID NO. |
| | (for AsCas12a) | 1 |
| | UAAUUUCUACUAAGUGUAGAUCCUCACCAUUUCCAUUUAC | SEQ ID NO. |
| | U (for LbCas12a) | 2 |
| SSDNA | AFTAAATGGAAATGGTGAGG | SEQ ID NO. |
| target | | 3 |
| ssRNA | AGUAAAUGGAAAUGGUGAGG | SEQ ID NO. |
| target | | 4 |
| SSDNA | /56-FAM/TTATT/3IABKFQ/ | SEQ ID NO. |
| reporter | | 5 |
| SSRNA | /56-FAM/UUUUU/3IABKFQ/ | SEQ ID NO. |
| reporter | | 6 |

TABLE 3
Sequences of nucleic acid targets used in gel electrophoresis for
analysis of cis-and trans-cleavage product. The crRNA sequence is the
same as used in the trans-cleavage assay. The underlined sequences
represent the targeted region.

| | | SEQ ID |
|---|---|---|
| Name | Sequence (5′-3′) | NO. |
| 5′ | AATAAGAGACAAGATAAGGGAGTAAATGGAAATGGTGAGG | SEQ ID |
| extended | | NO. 7 |
| SSDNA | | |
| target | | |
| 5′ | /56-FAM/AAUAAGAGACAAGAUAAGGGAGUAAAUGGAAAUGG | SEQ ID |
| extended | UGAGG | NO. 8 |
| SSRNA | | |
| target with | | |
| 5′ FAM- | | |
| labeling | | |
| 5′ and 3′ | /56-FAM/AAUAAGAGACAAGAUAAGGGAGUAAAUGGAAAUGG | SEQ ID |
| extended | UGAGGUAAGACUAGGUUAGACUGGA | NO. 9 |
| SSRNA | | |
| target with | | |
| 5′ FAM- | | |
| labeling | | |
| 5′ and 3′ | AAUAAGAGACAAGAUAAGGGAGUAAAUGGAAAUGGUGAGG | SEQ ID |
| extended SSRNA | UAAGACUAGGUUAGACUGGA/36-FAM/ | NO. 10 |
| target with | | |
| 3′ FAM- | | |
| labeling | | |

TABLE 4
Sequences used in the UNIVERSE assay for detection of HIV plasmid.
The underlined sequences are primers and their targeting regions. The
bold sequences are spacers of crRNA and its targeting region, except
GAAATTAATACGACTCACTATAGGG (SEQ ID NO: 42) in “RPA forward primer_
HIV” refers to the T7 promoter.

| Name | Sequence (5′-3′) | SEQ ID NO. |
|---|---|---|
| RPA forward | GAAATTAATACGACTCACTATAGGGAAGCAGCCATG | SEQ ID NO. 11 |
| primer_HIV | CAAATGTTAAAAGAGACCATC | |
| RPA reverse | GTAGTTCCTGCTATGTCACTTCCCCTTGGTTC | SEQ ID NO. 12 |
| primer_HIV | | |
| crRNA_ | UAAUUUCUACUCUUGUAGAUAUGCACUCUAUCCCAUU | SEQ ID NO. 13 |
| plasmid_HIV | CUG (for AsCas12a) | |
| | UAAUUUCUACUAAGUGUAGAUAUGCACUCUAUCCCAU | SEQ ID NO. 14 |
| | UCUG (for LbCas12a) | |
| HIV p24 | CCAGAAGTGATACCCATGTTTTCAGCATTATCAGAAG | SEQ ID NO. 15 |
| Plasmid | GAGCCACCCCACAAGATTTAAACACCATGCTAAACAC | |
| (NCBI | AGTGGGGGGACATCAAGCAGCCATGCAAATGTTAAAA | |
| Reference | GAGACCATCAATGAGGAAGCTGCAGAATGGGATAGAG | |
| Sequence: | TGCATCCAGTGCATGCAGGGCCTATTGCACCAGGCCA | |
| NC_001802.1) | GATGAGAGAACCAAGGGGAAGTGACATAGCAGGAACT | |
| | ACTAGTACCCTTCAGGAACAAATAGGATGGATGACAAA | |
| | TAATCCACCTATCCCAGTAGGAGAAATTTATAAAAGAT | |
| | GG | |

TABLE 5
Sequences used in the UNIVERSE assay and the RT-qPCR assay for detection of
positive control and clinical samples of HIV. The underlined sequences are
primers and their targeting regions. The bold sequences are spacers of
crRNA and its targeting region, except the sequence
GAAATTAATACGACTCACTATAGGG (SEQ ID NO: 42) in “RPA forward primer_HIV” and
in “RPA amplicon sequence” refers to the T7 promoter.

| Name | Sequence (5′-3′) | SEQ ID NO. |
|---|---|---|
| RPA forward | GAAATTAATACGACTCACTATAGGGAAGCAGCCATG | SEQ ID NO. |
| primer_HIV | CAAATGTTAAAAGAGACCATC | 16 |
| RPA reverse | GTAGTTCCTGCTATGTCACTTCCCCTTGGTTC | SEQ ID NO. |
| primer_HIV | | 17 |
| crRNA_clinical_ | UAAUUUCUACUAAGUGUAGAUCCUGGUGCAAUAG | SEQ ID NO. |
| HIV | GCCCUGC (for LbCas12a) | 18 |
| RPA amplicon | GAAATTAATACGACTCACTATAGGGAAGCAGCCATG | SEQ ID NO. |
| sequence | CAAATGTTAAAAGAGACCATCAATGAGGAAGCTGCA | 19 |
| (NCBI Reference | GAATGGGATAGAGTGCATCCAGTGCATGCAGGGCC | |
| Sequence: | TATTGCACCAGGCCAGATGAGAGAACCAAGGGGAA | |
| NC_001802.1) | GTGACATAGCAGGAACTA | |
| PCR forward | CAAGCAGCCATGCAAATGTTA | SEQ ID NO. |
| primer_HIV | | 20 |
| PCR reverse | TGTCATCCATCCTATTTGTTCCT | SEQ ID NO. |
| primer_HIV | | 21 |
| qPCR probe_HIV | /56- | SEQ ID NO. |
| | FAM/TTGGATCTC/ZEN/TTATCTGGCCTGGTGC/ | 22 |
| | 3IABkFQ/ | |
| PCR amplicon | CAAGCAGCCATGCAAATGTTAAAAGAGACCATCAAT | SEQ ID NO. |
| sequence | GAGGAAGCTGCAGAATGGGATAGAGTGCATCCAGT | 23 |
| (NCBI Reference | GCATGCAGGGCCTATTGCACCAGGCCAGATGAGAGA | |
| Sequence: | ACCAAGGGGAAGTGACATAGCAGGAACTACTAGTA | |
| NC_001802.1) | CCCTTCAGGAACAAATAGGATGGATGACA | |

TABLE 6
Sequences used in the UNIVERSE assay and the qPCR assay for detection of
plasmid and clinical samples of HPV 16. The underlined sequences are
primers and their targeting regions. The bold sequences are spacers of crRNA
and its targeting region, except the sequence GAAATTAATACGACTCACTATAGGG
(SEQ ID NO: 42) in “RPA forward primer_HIV” and in “RPA amplicon sequence”
refers to the T7 promoter.

| Name | Sequence (5′-3′) | SEQ ID NO. |
|---|---|---|
| RPA | GAAATTAATACGACTCACTATAGGGGCAAACCACCTATA | SEQ ID NO. 24 |
| forward | GGGGAACACTGGGGCAAAG | |
| primer_HPV16 | | |
| RPA reverse | TAGATGTACAAATATCCAGTGGAACTTCAC | SEQ ID NO. 25 |
| primer_HPV16 | | |
| crRNA_ | UAAUUUCUACUAAGUGUAGAUCUGGAUUUACUGCAACAU | SEQ ID NO. 26 |
| clinical_ | UG (for LbCas12a) | |
| HPV16 | | |
| RPA | GAAATTAATACCACTCACTATAGGGGCAAACCACCTATA | SEQ ID NO. 27 |
| amplicon | GGGGAACACTGGGGCAAAGGATCCCCATGTACCAATGTTG | |
| sequence | CAGTAAATCCAGGTGATTGTCCACCATTAGAGTTAATAAA | |
| (NCBI | CACAGTTATTCAGGATGGTGATATGGTTGATACTGGCTTTG | |
| Reference | GTGCTATGGACTTTACTACATTACAGGCTAACAAAAGTGAA | |
| Sequence: | GTTCCACTGGATATTTGTACATCTA | |
| NC_001526.4) | | |
| HPV 16 L1 | GTTCCTAAAGTATCAGGATTACAATACAGGGTATTTAGAAT | SEQ ID NO. 28 |
| plasmid | ACATTTACCTGACCCCAATAAGTTTGGTTTTCCTGACACCTC | |
| (NCBI | ATTTTATAATCCAGATACACAGCGGCTGGTTTGGGCCTGTG | |
| Reference | TAGGTGTTGAGGTAGGTCGTGGTCAGCCATTAGGTGTGGGC | |
| Sequence: | ATTAGTGGCCATCCTTTATTAAATAAATTGGATGACACAGA | |
| NC_001526.4)) | AAATGCTAGTGCTTATGCAGCAAATGCAGGTGTGGATAAT | |
| | AGAGAATGTATATCTATGGATTACAAACAAACACAATTGT | |
| | GTTTAATTGGTTGCAAACCACCTATAGGGGAACACTGGGGC | |
| | AAAGGATCCCCATGTACCAATGTTGCAGTAAATCCAGGT | |
| | GATTGTCCACCATTAGAGTTAATAAACACAGTTATTCAGGA | |
| | TGGTGATATGGTTGATACTGGCTTTGGTGCTATGGACTTTA | |
| | CTACATTACAGGCTAACAAAAGTGAAGTTCCACTGGATATT | |
| | TGTACATCTA | |
| PCR | ACTGTTGTTGATACTACACGCA | SEQ ID NO. 29 |
| forward | | |
| primer_HPV16 | | |
| PCR reverse | GTCGTAGGTACTCCTTAAAGTTAGTATT | SEQ ID NO. 30 |
| primer_HPV16 | | |
| qPCR | /56FAM/TGTGCTGCC/ZEN/ATATCTACTTCAGAAACTACA/ | SEQ ID NO. 31 |
| probe_HPV16 | 3IABKFQ/ | |
| PCR | ACTGTTGTTGATACTACACGCAGTACAAATATGTCATTATG | SEQ ID NO. 32 |
| amplicon | TGCTGCCATATCTACTTCAGAAACTACATATAAAAATACTA | |
| sequence | ACTTTAAGGAGTACCTACGAC | |
| (NCBI | | |
| Reference | | |
| Sequence: | | |
| NC_001526.4) | | |

TABLE 7
Sequences of the RPA primers and crRNAs used for detection of arbitrary sequences
in the HIV amplicon using the UNIVERSE assay. The underlined sequences are
primers and their targeting regions. The bold sequences are spacers of crRNA and
its targeting region, except the sequence CAAATTAATACGACTCACTATAGGG (SEQ ID NO: 42)
in “RPA forward primer_HIV” and in “RPA amplicon sequence” refers to the
T7 promoter. All crRNAs are for LbCas12a.

| Name | Sequence (5′-3′) | SEQ ID NO. |
|---|---|---|
| RPA | GAAATTAATACGACTCACTATAGGGAAGCAGCCATGC | SEQ ID NO. 33 |
| forward | AAATGTTAAAAGAGACCATC | |
| primer_HIV | | |
| RPA reverse | GTAGTTCCTGCTATGTCACTTCCCCTTGGTTC | SEQ ID NO. 34 |
| primer_HIV | | |
| crRNA-1 | UAAUUUCUACUAAGUGUAGAUUGCACUGGAUGCACUCU | SEQ ID NO. 35 |
| | AUC | |
| crRNA-2 | UAAUUUCUACUAAGUGUAGAUAGCCCCUGCAUGCACUG | SEQ ID NO. 36 |
| | GAU | |
| crRNA-3 | UAAUUUCUACUAAGUGUAGAUUGCAAUAGGCCCUGCAU | SEQ ID NO. 37 |
| | GCA | |
| crRNA-4 | UAAUUUCUACUAAGUGUAGAUCCUGGUGCAAUAGGCCC | SEQ ID NO. 38 |
| | UGC | |
| crRNA-5 | UAAUUUCUACUAAGUGUAGAUUCUGGCCUGGUGCAAUA | SEQ ID NO. 39 |
| | GGC | |
| crRNA-6 | UAAUUUCUACUAAGUGUAGAUCCCCUUGGUUCUCUCAU | SEQ ID NO. 40 |
| | CUG | |
| RPA | GAAATTAATACGACTCACTATAGGGAAGCAGCCATGC | SEQ ID NO. 41 |
| amplicon | AAATGTTAAAAGAGACCATCAATGAGGAAGCTGCAGAA | |
| sequence | TGGGATAGAGTGCATCCAGTGCATGCAGGGCCTATTGC | |
| (NCBI | ACCAGGCCAGATGAGAGAACCAAGGGGAAGTGACATAG | |
| Reference | CAGGAACTA | |
| Sequence: | | |
| NC_001802.1) | | |

For reasons of completeness, various aspects of the disclosure are set out in the following numbered clauses:

Clause 1. A method for detecting a microbial nucleic acid in a sample, which method comprises:
A) obtaining a sample suspected of containing the microbial nucleic acid from a subject;
B) generating amplicons of the microbial nucleic acid via contacting the sample suspected of containing the microbial nucleic acid with recombinase polymerase and reagents for amplification of the microbial nucleic acid and amplifying the microbial nucleic acid via recombinase polymerase amplification (RPA), wherein the amplicons of the microbial nucleic acid lack protospacer adjacent motif (PAM) sequences;
C) transcribing the amplicons of the microbial nucleic acid into single-stranded RNA (ssRNA) targets using T7 RNA polymerase;
D) incubating the ssRNA targets with a CRISPR-Cas12a nuclease, one or more guide RNA sequences specific for one or more regions within the ssRNA targets, and reporter molecules;
E) activating the CRISPR-Cas12a nuclease via binding of the one or more guide RNA sequences to the one or more regions within the ssRNA targets; and
F) generating a detectable signal via CRISPR-Cas12a trans-cleavage of the reporter molecules;
wherein the presence of a detectable signal indicates the presence of the microbial nucleic acid in the sample, and the absence of detectable signal indicates the absence of the microbial nucleic acid in the sample.

Clause 2. The method of clause 1, wherein the method detects attomolar amounts of microbial nucleic acid.

Clause 3. The method of clause 1, wherein activating the CRISPR-Cas12a nuclease does not occur when there is a mismatch between the ssRNA targets and the guide RNA sequences.

Clause 4. The method of clause 3, wherein the mismatch is a single base-pair difference.

Clause 5. The method of clause 1, wherein activating the CRISPR-Cas12a nuclease occurs via binding of the one or more guide RNA sequences to the one or more regions within the ssRNA targets.

Clause 6. The method of clause 1, wherein the sample comprises blood, mucous, serum, plasma, saliva, urine, stool, vaginal fluid, synovial fluid, spinal fluid, and/or semen.

Clause 7. The method of clause 1, wherein the reporter molecule is a single-stranded DNA (ssDNA) probe tagged with a fluorophore and quencher.

Clause 8. The method of clause 1, wherein the microbial nucleic acid is from a pathogenic virus.

Clause 9. The method of clause 1, wherein the microbial nucleic acid is from a pathogenic bacteria.

Clause 10. The method of clause 1, wherein the microbial nucleic acid is from a pathogenic fungus.

Clause 11. The method of clause 1, wherein the CRISPR-Cas12a nuclease is AsCas12a.

Clause 12. The method of clause 1, wherein the CRISPR-Cas12a nuclease is an AsCas12a variant.

Clause 13. The method of clause 1, wherein the CRISPR-Cas12a nuclease is AsCas12a V3.

Clause 14. The method of clause 1, wherein the CRISPR-Cas12a nuclease is AsCas12a Ultra.

Clause 15. The method of clause 14, wherein the threshold of detection of the method is about 3 copies/μL.

Clause 16. The method of clause 1, wherein the CRISPR-Cas12a nuclease is AsCas12a, AsCas12a V3, AsCas12a Ultra, LbCas12a, or combination thereof.

Clause 17. The method of clause 1, wherein incubating comprises conditions sufficient to allow binding of the one or more guide RNA sequences to the one or more regions within the ssRNA targets.

Clause 18. The method of clause 1, wherein the method uses one or more control sequences to monitor and/or verify reaction efficiency and/or to act as a positive control or negative control.

Clause 19. The method of clause 18, wherein the control sequence is a synthetic RNA or DNA control target sequence.

Clause 20. The method of clause 1, wherein the method is used for parallel, simultaneous detection ssRNA targets from a plurality of different pathogenic microbes.

Clause 21. The method of clause 20, wherein the plurality of different pathogenic microbes are a plurality of viruses, bacteria, protozoa, and/or fungi.

Clause 22. The method of clause 1, wherein steps B through F occur in a single vessel or tube.

Clause 23. The method of clause 1, wherein prior to step B, nucleic acid is extracted and/or purified from the sample.

Clause 24. The method of clause 1, wherein generating amplicons of the microbial nucleic acid comprises use of a primer comprising a T7 promoter sequence.

Clause 25. The method of clause 1, wherein the activated CRISPR-Cas12a nuclease does not cleave the ssRNA targets.

Clause 26. The method of clause 1, wherein the microbial nucleic acid is DNA.

Clause 27. The method of clause 1, wherein the microbial nucleic acid is RNA.

Clause 28. The method of clause 27, wherein, reverse transcription is utilized to generate cDNA.

Clause 29. The method of clause 28, wherein reverse transcription and step (B) occur at the same time in the same vessel or tube.

Clause 30. The method of clause 1, wherein the method is used in a lateral flow immunochromatographic assay.

Clause 31. The method of clause 1, wherein the method is performed more than one time from different samples obtained from the subject over a period of time.

Clause 32. A kit for detecting a microbial nucleic acid in a sample comprising:
A) recombinase polymerase;
B) a primer comprising a T7 promoter sequence;
C) reagents for amplification of the microbial nucleic acid sufficient to generate amplicons of the microbial nucleic acid lacking protospacer adjacent motif (PAM) sequences;
D) T7 RNA polymerase for transcribing amplicons of the microbial nucleic acid into single-stranded RNA (ssRNA) targets;
E) CRISPR-Cas12a nuclease;
F) one or more guide RNA sequences specific for one or more regions within the ssRNA targets; and
G) reporter molecules.

Clause 33. The kit of clause 32, wherein the reporter molecules comprise SEQ ID NO. 5. Clause 34. The kit of clause 32, wherein the reporter molecules comprise SEQ ID NO. 6.

Clause 35. The kit of clause 32, wherein the primer comprising a T7 promoter sequence comprises SEQ ID NO. 11, SEQ ID NO. 16, SEQ ID NO. 24, or SEQ ID NO. 33.

Clause 36. The kit of clause 32, wherein other reagents for amplification of the microbial nucleic acid sufficient to generate amplicons of the microbial nucleic acid lacking protospacer adjacent motif (PAM) sequences comprise SEQ ID NO. 12, SEQ ID NO. 17, SEQ ID NO. 25, or SEQ ID NO. 34.

Clause 37. The kit of clause 32, wherein the one or more guide RNA sequences comprise SEQ ID NO. 13, SEQ ID NO. 18, SEQ ID NO. 26, SEQ ID NO. 35, SEQ ID NO. 36, SEQ ID NO. 37, SEQ ID NO. 38, SEQ ID NO. 39, or SEQ ID NO. 40.

Clause 38. The kit of clause 32, further comprising a control target sequence comprising SEQ ID NO. 14, SEQ ID NO. 19, SEQ ID NO. 27, SEQ ID NO. 28, or SEQ ID NO. 41.

Embodiments disclosed here are not limiting of the subject matter and is merely exemplary. Various other components may be included and called upon for providing for aspects of the teachings herein. For example, additional materials, combinations of materials and/or omission of materials may be used to provide for added embodiments that are within the scope of the teachings herein.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method for detecting a microbial nucleic acid in a sample, which method comprises:
A) obtaining a sample suspected of containing the microbial nucleic acid from a subject;
B) generating amplicons of the microbial nucleic acid via contacting the sample suspected of containing the microbial nucleic acid with recombinase polymerase and reagents for amplification of the microbial nucleic acid and amplifying the microbial nucleic acid via recombinase polymerase amplification (RPA), wherein the amplicons of the microbial nucleic acid lack protospacer adjacent motif (PAM) sequences;
C) transcribing the amplicons of the microbial nucleic acid into single-stranded RNA (ssRNA) targets using T7 RNA polymerase;
D) incubating the ssRNA targets with a CRISPR-Cas12a nuclease, one or more guide RNA sequences specific for one or more regions within the ssRNA targets, and reporter molecules;
E) activating the CRISPR-Cas12a nuclease via binding of the one or more guide RNA sequences to the one or more regions within the ssRNA targets; and
F) generating a detectable signal via CRISPR-Cas12a trans-cleavage of the reporter molecules; wherein the presence of a detectable signal indicates the presence of the microbial nucleic acid in the sample, and the absence of detectable signal indicates the absence of the microbial nucleic acid in the sample.

2. The method of claim 1, wherein activating the CRISPR-Cas12a nuclease does not occur when there is a single base-pair mismatch between the ssRNA targets and the guide RNA sequences.

3. The method of claim 1, wherein the sample comprises blood, mucous, serum, plasma, saliva, urine, stool, vaginal fluid, synovial fluid, spinal fluid, and/or semen.

4. The method of claim 1, wherein the reporter molecule is a single-stranded DNA (ssDNA) probe tagged with a fluorophore and quencher.

5. The method of claim 1, wherein the microbial nucleic acid is from a pathogenic virus.

6. The method of claim 1, wherein the microbial nucleic acid is from a pathogenic bacteria.

7. The method of claim 1, wherein the microbial nucleic acid is from a pathogenic fungus.

8. The method of claim 1, wherein the CRISPR-Cas12a nuclease is AsCas12a, AsCas12a V3, AsCas12a Ultra, LbCas12a, or combination thereof.

9. The method of claim 1, wherein the CRISPR-Cas12a nuclease is AsCas12a Ultra.

10. The method of claim 9, wherein the threshold of detection of the method is about 3 copies/μL.

11. The method of claim 1, wherein steps B through F occur in a single vessel or tube.

12. The method of claim 1, wherein the activated CRISPR-Cas12a nuclease does not cleave the ssRNA targets.

13. The method of claim 1, wherein the method is used for parallel, simultaneous detection of ssRNA targets from a plurality of different pathogenic microbes.

14. The method of claim 1, wherein prior to step B, nucleic acid is extracted and/or purified from the sample.

15. The method of claim 1, wherein generating amplicons of the microbial nucleic acid comprises use of a primer comprising a T7 promoter sequence.

16. The method of claim 1, wherein the microbial nucleic acid is RNA and reverse transcription is utilized to generate cDNA, and wherein the reverse transcription and step (B) occur at the same time in the same vessel or tube.

17. A kit for detecting a microbial nucleic acid in a sample comprising:
A) recombinase polymerase;
B) a primer comprising a T7 promoter sequence;
C) reagents for amplification of the microbial nucleic acid sufficient to generate amplicons of the microbial nucleic acid lacking protospacer adjacent motif (PAM) sequences;
D) T7 RNA polymerase for transcribing amplicons of the microbial nucleic acid into single-stranded RNA (ssRNA) targets;
E) CRISPR-Cas12a nuclease;
F) one or more guide RNA sequences specific for one or more regions within the ssRNA targets; and
G) reporter molecules.

18. The kit of claim 17, wherein the reporter molecules comprise SEQ ID NO. 5 and/or SEQ ID NO. 6.

19. The kit of claim 17, wherein the primer comprising a T7 promoter sequence comprises SEQ ID NO. 11, SEQ ID NO. 16, SEQ ID NO. 24, or SEQ ID NO. 33; the other reagents for amplification of the microbial nucleic acid sufficient to generate amplicons of the microbial nucleic acid lacking protospacer adjacent motif (PAM) sequences comprise SEQ ID NO. 12, SEQ ID NO. 17, SEQ ID NO. 25, or SEQ ID NO. 34; and/or the one or more guide RNA sequences comprise SEQ ID NO. 13, SEQ ID NO. 18, SEQ ID NO. 26, SEQ ID NO. 35, SEQ ID NO. 36, SEQ ID NO. 37, SEQ ID NO. 38, SEQ ID NO. 39, or SEQ ID NO. 40.

20. The kit of claim 17, further comprising a control target sequence comprising SEQ ID NO. 14, SEQ ID NO. 19, SEQ ID NO. 27, SEQ ID NO. 28, or SEQ ID NO. 41.


SEQUENCE LISTING (42 sequences)

SEQ ID NO: 1 - RNA, 40 nt, Synthetic construct
  source 1..40: mol_type = other RNA
taatttctactcttgtagatcctcaccatttccatttact

SEQ ID NO: 2 - RNA, 41 nt, Synthetic construct
  source 1..41: mol_type = other RNA
taatttctactaagtgtagatcctcaccatttccatttact

SEQ ID NO: 3 - DNA, 20 bp, Synthetic construct
  source 1..20: mol_type = other DNA
agtaaatggaaatggtgagg

SEQ ID NO: 4 - RNA, 20 nt, Synthetic construct
  source 1..20: mol_type = other RNA
agtaaatggaaatggtgagg

SEQ ID NO: 5-6 - skipped (2 entries)

SEQ ID NO: 7 - DNA, 40 bp, Synthetic construct
  source 1..40: mol_type = other DNA
aataagagacaagataagggagtaaatggaaatggtgagg

SEQ ID NO: 8 - RNA, 35 nt, Synthetic construct
  source 1..35: mol_type = other RNA
aataagagacaagataagggagtaaatggaaatgg

SEQ ID NO: 9 - RNA, 60 nt, Synthetic construct
  source 1..60: mol_type = other RNA
aataagagacaagataagggagtaaatggaaatggtgaggtaagactaggttagactgga

SEQ ID NO: 10 - RNA, 60 nt, Synthetic construct; identical to SEQ ID NO: 9
  source 1..60: mol_type = other RNA

SEQ ID NO: 11 - DNA, 57 bp, Synthetic construct
  source 1..57: mol_type = other DNA
gaaattaatacgactcactatagggaagcagccatgcaaatgttaaaagagaccatc

SEQ ID NO: 12 - DNA, 32 bp, Synthetic construct
  source 1..32: mol_type = other DNA
gtagttcctgctatgtcacttccccttggttc

SEQ ID NO: 13 - RNA, 40 nt, Synthetic construct
  source 1..40: mol_type = other RNA
taatttctactcttgtagatatgcactctatcccattctg

SEQ ID NO: 14 - RNA, 41 nt, Synthetic construct
  source 1..41: mol_type = other RNA
taatttctactaagtgtagatatgcactctatcccattctg

SEQ ID NO: 15 - DNA, 300 bp, Synthetic construct
  source 1..300: mol_type = other DNA
ccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatgg

SEQ ID NO: 16 - DNA, 57 bp, Synthetic construct; identical to SEQ ID NO: 11
  source 1..57: mol_type = other DNA

SEQ ID NO: 17 - DNA, 32 bp, Synthetic construct; identical to SEQ ID NO: 12
  source 1..32: mol_type = other DNA

SEQ ID NO: 18 - RNA, 41 nt, Synthetic construct
  source 1..41: mol_type = other RNA
taatttctactaagtgtagatcctggtgcaataggccctgc

SEQ ID NO: 19 - DNA, 160 bp, Synthetic construct
  source 1..160: mol_type = other DNA
gaaattaatacgactcactatagggaagcagccatgcaaatgttaaaagagaccatcaatgaggaagctgcagaatgggatagagtgcatccagtgcatgcagggcctattgcaccaggccagatgagagaaccaaggggaagtgacatagcaggaacta

SEQ ID NO: 20 - DNA, 21 bp, Synthetic construct
  source 1..21: mol_type = other DNA
caagcagccatgcaaatgtta

SEQ ID NO: 21 - DNA, 23 bp, Synthetic construct
  source 1..23: mol_type = other DNA
tgtcatccatcctatttgttcct

SEQ ID NO: 22 - DNA, 25 bp, Synthetic construct
  source 1..25: mol_type = other DNA
ttggatctcttatctggcctggtgc

SEQ ID NO: 23 - DNA, 171 bp, Synthetic construct
  source 1..171: mol_type = other DNA
caagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgaca

SEQ ID NO: 24 - DNA, 58 bp, Synthetic construct
  source 1..58: mol_type = other DNA
gaaattaatacgactcactataggggcaaaccacctataggggaacactggggcaaag

SEQ ID NO: 25 - DNA, 30 bp, Synthetic construct
  source 1..30: mol_type = other DNA
tagatgtacaaatatccagtggaacttcac

SEQ ID NO: 26 - RNA, 41 nt, Synthetic construct
  source 1..41: mol_type = other RNA
taatttctactaagtgtagatctggatttactgcaacattg

SEQ ID NO: 27 - DNA, 226 bp, Synthetic construct
  source 1..226: mol_type = other DNA
gaaattaatacgactcactataggggcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatcta

SEQ ID NO: 28 - DNA, 499 bp, Synthetic construct
  source 1..499: mol_type = other DNA
gttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttgatactggctttggtgctatggactttactacattacaggctaacaaaagtgaagttccactggatatttgtacatcta

SEQ ID NO: 29 - DNA, 22 bp, Synthetic construct
  source 1..22: mol_type = other DNA
actgttgttgatactacacgca

SEQ ID NO: 30 - DNA, 28 bp, Synthetic construct
  source 1..28: mol_type = other DNA
gtcgtaggtactccttaaagttagtatt

SEQ ID NO: 31 - DNA, 30 bp, Synthetic construct
  source 1..30: mol_type = other DNA
tgtgctgccatatctacttcagaaactaca

SEQ ID NO: 32 - DNA, 103 bp, Synthetic construct
  source 1..103: mol_type = other DNA
actgttgttgatactacacgcagtacaaatatgtcattatgtgctgccatatctacttcagaaactacatataaaaatactaactttaaggagtacctacgac

SEQ ID NO: 33 - DNA, 57 bp, Synthetic construct; identical to SEQ ID NO: 11
  source 1..57: mol_type = other DNA

SEQ ID NO: 34 - DNA, 32 bp, Synthetic construct; identical to SEQ ID NO: 12
  source 1..32: mol_type = other DNA

SEQ ID NO: 35 - RNA, 41 nt, Synthetic construct
  source 1..41: mol_type = other RNA
taatttctactaagtgtagattgcactggatgcactctatc

SEQ ID NO: 36 - RNA, 41 nt, Synthetic construct
  source 1..41: mol_type = other RNA
taatttctactaagtgtagataggccctgcatgcactggat

SEQ ID NO: 37 - RNA, 41 nt, Synthetic construct
  source 1..41: mol_type = other RNA
taatttctactaagtgtagattgcaataggccctgcatgca

SEQ ID NO: 38 - RNA, 41 nt, Synthetic construct; identical to SEQ ID NO: 18
  source 1..41: mol_type = other RNA

SEQ ID NO: 39 - RNA, 41 nt, Synthetic construct
  source 1..41: mol_type = other RNA
taatttctactaagtgtagattctggcctggtgcaataggc

SEQ ID NO: 40 - RNA, 41 nt, Synthetic construct
  source 1..41: mol_type = other RNA
taatttctactaagtgtagatccccttggttctctcatctg

SEQ ID NO: 41 - DNA, 160 bp, Synthetic construct; identical to SEQ ID NO: 19
  source 1..160: mol_type = other DNA

SEQ ID NO: 42 - DNA, 25 bp, Synthetic construct
  source 1..25: mol_type = other DNA
gaaattaatacgactcactataggg